(19) United States
(12) Patent Application Publication
(10) Pub. No.: US 2021/0171885 A1
(43) Pub. Date: Jun. 10, 2021

(54) METHOD FOR PRODUCING CELL AGGREGATES

(71) Applicants: KANEKA CORPORATION, Osaka (JP); The University of Tokyo, Tokyo (JP)

(72) Inventors: Ikki Horiguchi, Tokyo (JP); Yasuyuki Sakai, Tokyo (JP); Masato Ibuki, Hyogo (JP)

(73) Assignees: KANEKA CORPORATION, Osaka (JP); The University of Tokyo, Tokyo (JP)

(21) Appl. No.: 17/178,987

(22) Filed: Feb. 18, 2021

Related U.S. Application Data
(63) Continuation of application No. 15/663,241, filed on Jul. 28, 2017, now abandoned.
Application No. 15/663,241 is a continuation of application No. PCT/JP2016/052128, filed on Jan. 26, 2016.

(30) Foreign Application Priority Data
Jan. 29, 2015 (JP) 2015-015306

(51) Int. Cl. C12M 3/02; C12N 5/10; C12N 5/00

(52) U.S. Cl. CPC C12M 3/02; C12N 5/10; C12N 2525/00; C12N 2500/72; C12N 5/0018

(57) ABSTRACT

A method for producing cell aggregates includes culturing cells while suspending the cells in a liquid culture medium comprising a lysophospholipid. A composition includes a lysophospholipid, wherein the composition is a liquid culture medium or a composition added to a liquid culture medium.

### TECHNICAL FIELD

One or more embodiments of the present invention relate to a method for producing cell aggregates such that useful cells such as pluripotent stem cells are cultured in suspension in a liquid culture medium.

### BACKGROUND

Recent research on human pluripotent stem cells (e.g., human ES cells and human iPS cells) has increasingly made regenerative medicine come in reality. These cells possess an ability to proliferate infinitely and an ability to differentiate into various types of cells. Thus, regenerative medicine using the pluripotent stem cells should radically change therapeutic interventions against, for example, refractory diseases and lifestyle-related diseases. It has already been possible that the pluripotent stem cells can be induced and differentiated in vitro into various types of cells including neurons, cardiomyocytes, blood cells, and retinal cells.

One of objectives directed toward practical use of regenerative medicine in which pluripotent stem cells are used to regenerate a variety of organs involves how a large number of cells necessary for regeneration of organs can be produced efficiently. For example, the regeneration of a liver requires about 2×10¹¹ cells. A substrate plate with an area of 10⁶ cm² or more is needed so as to culture the above number of cells using adherent culture on a flat substrate plate. This means that about 20,000 common 10-cm dishes are needed. Because the number of cells to be obtained using adherent culture on a surface of the substrate plate depends on the surface area of the culture plate, it is difficult to scale up the culture. Accordingly, it is hard to provide an enough number of cells to make regenerative medicine available.

It is easy to scale up suspension culture in which cells are cultured in suspension in a liquid culture medium. Hence, the suspension culture should be fit for mass production of cells.

Non-Patent Literature 3 discloses a process for producing spheroids with a uniform size, the process comprising: using a spinner flask as cell cultureware for suspension culture; and culturing human pluripotent stem cells in suspension while strongly stirring a liquid culture medium.

Non-Patent Literature 4 discloses a process for producing spheroids with a uniform size in each micro-well, the process comprising using a substrate plate on which small micro-wells are formed.

Non-Patent Literature 5 discloses a culturing method comprising: using a culture medium the viscosity and specific gravity of which is adjusted; keeping pluripotent stem cells in suspension; and reducing a collision between the cells.

Patent Literature 1 discloses a technology in which cells are cultured while being subjected to rotary shaking culture in a liquid culture medium, so that cell aggregates are produced.

Patent Literature 2 discloses a method in which pluripotent stem cells are cultured in suspension until the average diameter of cell aggregates reaches about 200 to 300 μm.

### CITATION LIST

### Patent Literature

Patent Literature 1: JP Patent Publication (Kokai) No. 2003-304866A
Patent Literature 2: WO2013/077423A

### Non-Patent Literature

Non-Patent Literature 1: Takayama N, Nishimura S, Nakamura S, Shimizu T, Ohnishi R, Endo H, Yamaguchi T, Otsu M, Nishimura K, Nakanishi M, Sawaguchi A, Nagai R, Takahashi K, Yamanaka S, Nakauchi H, Eto K. Transient activation of c-MYC expression is critical for efficient platelet generation from human induced pluripotent stem cells. J Exp Med. 2010 Dec. 20; 207(13): 2817-30. doi: 10.1084/jem.20100844. Epub 2010 Nov. 22. PubMed PMID: 21098095; PubMed Central PMCID: PMC3005234.
Non-Patent Literature 2: F R Garcia-Gonzalo and JCI Belmonte, Albumin-Associated Lipids Regulate Human Embryonic Stem Cell Self-Renewal. PLoS ONE, 2008, 3(1), e1384
Non-Patent Literature 3: Olmer R. et al., Tissue Engineering: Part C, Volume 18 (10): 772-784 (2012)
Non-Patent Literature 4: Ungrin M D et al., PLoS ONE, 2008, 3(2), e1565
Non-Patent Literature 5: Otsuji G T et al., Stem Cell Reports, Volume 2: 734-745 (2014)

### SUMMARY

As described above, various kinds of technology for preparing cell aggregates using suspension culture have been developed. However, the present inventors have thought of there still being disadvantages of these kinds of technology.

Adherent cells (e.g., pluripotent stem cells) can form aggregates while cultured in suspension. Examples of the mechanism of cell aggregation include: non-specific cell-to-cell attachment mediated by membrane proteins and/or plasma membranes; and intercellular adhesion mediated by cadherins on the cell surface. Because cells such as human iPS cells cannot survive as single cells, it is necessary for the cells to form cell aggregates for survival. However, when the size of the aggregates is too large, nutrients cannot be sufficiently distributed into cells located deep inside the aggregates, which may cause inhibition of their proliferation and difficulty to maintain an undifferentiated state. To prevent excessive aggregation, it seems effective to make a liquid culture medium flow during culture. The excessive flow, however, could damage cells due to physical stimulation to cells. Here, a suspension culture technology has been sought that is used to produce aggregates with an appropriate size without damaging cells. The conventional methods for producing aggregates listed in the Background section still have room for improvement.

The process of Non-Patent Literature 3 likely causes cells to die due to shear stress, which is a defect of the process.

In the process of Non-Patent Literature 4, it is difficult to scale up a culture and to change a culture medium.

In the method of Non-Patent Literature 5, because of less movement of a culture medium during culture, nutritional components are less likely to be supplied to cell aggregates.

Patent Literature 1 fails to disclose a means for controlling the size of cell aggregates to an appropriate size.

Patent Literature 2 discloses adding, to a culture medium, an aqueous polymer as a means for preventing adhesion between cell aggregates, so that the viscosity increases. This causes the same defect as in the case of Non-Patent Literature 5, in which oxygen and nutritional components are less likely to be supplied to cell aggregates.

One or more embodiments of the present invention provide a method for producing cell aggregates using suspension culture, wherein it is easy to control the size of the cell aggregates so as to be appropriate for culturing, and the likelihood of damaging the cells is low.

The present inventors have obtained a surprising finding where when a lipid (e.g., a phospholipid) is added to a liquid culture medium and cells are cultured in suspension in the medium, a large amount of population of cell aggregates with an appropriate size can be produced. Based on this finding, the present inventors have completed one or more embodiments of the present invention. One or more embodiments of the present invention do not rely on mechanical/physical means, i.e., modifying culturing conditions, such as the viscosity of a culture medium, shear stress due to stirring, or the shape of cultureware such as a microwell plate. One or more embodiments of the present invention use biochemical/chemical means, i.e., modifying the composition of a culture medium by using a substance present in vivo. Specifically, one or more embodiments of the present invention encompass the following aspects.

(1) A method for producing cell aggregates, comprising a step of culturing cells while suspended in a liquid culture medium comprising a lysophospholipid. The step involves culturing cells while suspended in a liquid culture medium containing a lysophospholipid, so that an increase in the size of the cell aggregates in the culture is controlled.
(2) The method according to item (1), wherein the liquid culture medium comprises the lysophospholipid in an amount greater than 0.0064 μg/mL to 100 μg/mL.
(3) The method according to item (1) or (2), further comprising at least one of a step of adding the lysophospholipid to prepare the liquid culture medium and a step of generating the lysophospholipid through an enzymatic reaction to prepare the liquid culture medium.
(4) The method according to any one of items (1) to (3), wherein the cells are cells isolated after undergoing adherent or suspension culture.
(5) The method according to any one of items (1) to (4), wherein the lysophospholipid is at least one of lysophosphatidic acid and sphingosine-1-phosphoric acid.
(6) The method according to any one of items (1) to (5), wherein the liquid culture medium comprises at least one selected from the group consisting of L-ascorbic acid, insulin, transferrin, selenium, and sodium bicarbonate.
(7) The method according to any one of items (1) to (6), wherein the liquid culture medium comprises a growth factor.
(8) The method according to item (7), wherein the growth factor is at least one of FGF2 and TGF-β1.
(9) The method according to any one of items (1) to (8), wherein the liquid culture medium comprises a ROCK inhibitor.
(10) The method according to item (9), wherein the ROCK inhibitor is Y-27632.
(11) The method according to any one of items (1) to (10), wherein the cells are pluripotent stem cells.
(12) A cell aggregate produced by the method according to any one of items (1) to (11).
(13) A cell aggregation inhibitor comprising a lysophospholipid. In one or more embodiments of the present invention, the term “cell aggregation inhibitor” may be referred to as an “agent for controlling cell aggregation”.
(14) The cell aggregation inhibitor according to item (13), wherein the cell aggregation inhibitor comprises the lysophospholipid in an amount greater than 0.0064 μg/mL to 100 μg/mL.
(15) The cell aggregation inhibitor according to item (13) or (14), further comprising a growth factor.
(16) The cell aggregation inhibitor according to item (15), wherein the growth factor is at least one of FGF2 and TGF-β1.
(17) The cell aggregation inhibitor according to any one of items (13) to (16), further comprising a ROCK inhibitor.
(18) The cell aggregation inhibitor according to item (17), wherein the ROCK inhibitor is Y-27632.
(19) The cell aggregation inhibitor according to any one of items (13) to (18), wherein the lysophospholipid is at least one of lysophosphatidic acid and sphingosine-1-phosphoric acid.
(20) Use of a lysophospholipid for inhibition of cell aggregation. In one or more embodiments of the present invention, the wording “use for inhibition of cell aggregation” may be expressed as “use for control of cell aggregation in a cell culture”.
(21) The use according to item (20), wherein the lysophospholipid is present in a concentration greater than 0.0064 μg/mL to 100 μg/mL.
(22) The use according to item (20) or (21), wherein the lysophospholipid is used in combination with a growth factor.
(23) The use according to item (22), wherein the growth factor is at least one of FGF2 and TGF-β1.
(24) The use according to any one of items (20) to (23), wherein the lysophospholipid is used in combination with a ROCK inhibitor.
(25) The use according to item (24), wherein the ROCK inhibitor is Y-27632.
(26) The use according to any one of items (20) to (25), wherein the lysophospholipid is at least one of lysophosphatidic acid and sphingosine-1-phosphoric acid.
(27) A method for producing cell aggregates, comprising a step of culturing cells while suspended in a liquid culture medium comprising a lipid that has an ability to bind to albumin.
(28) The method according to item (27), wherein the liquid culture medium comprises the lipid in an amount of 0.00325 to 0.065 mg/ml.
(29) The method according to item (27) or (28), wherein the liquid culture medium comprises L-ascorbic acid, and/or insulin, and/or transferrin, and/or selenium, and/or sodium bicarbonate, and/or at least one growth factor.
(30) The method according to item (29), wherein the growth factor contained in the liquid culture medium is FGF2 and/or TGF-β1.
(31) The method according to any one of items (27) to (30), wherein the cells are pluripotent stem cells.
(32) A cell aggregate produced by the method according to any one of items (27) to (31).
(33) A cell aggregation inhibitor (or an agent for controlling cell aggregation) comprising a lipid that has an ability to bind to albumin.
(34) Use of a lipid that has an ability to bind to albumin for inhibition (or control) of cell aggregation.
(35) The cell aggregation inhibitor according to item (33) or the use according to item (34), wherein the lipid is present in a concentration of 0.00325 to 0.065 mg/ml.
(36) The cell aggregation inhibitor according to item (33) or the use according to item (34), wherein the lipid is used in combination with L-ascorbic acid, and/or insulin, and/or transferrin, and/or selenium, and/or sodium bicarbonate, and/or at least one growth factor.
(37) The cell aggregation inhibitor according to item (33) or the use according to item (34), wherein the cell is a pluripotent stem cell.

The text of specification includes disclosure of JP Patent Application No. 2015-015306, of which the present application claims priority.

The method of one or more embodiments of the present invention allows for mass production of cell aggregates that are fit for suspension culture and makes it possible to produce a large number of relevant cells.

### BRIEF DESCRIPTION OF THE DRAWINGS

FIG. 1 schematically illustrates an outline of a protocol for producing cell aggregates.

FIG. 2 shows three-dimensional aggregates that were produced from human iPS cells by rotary shaking culture with different KSR concentrations in a container non-adherent to cells (at Day 2 of culture). %=volume (v)/volume (v).

FIG. 3 shows the time course of change in glucose consumption when KSR at each concentration was added. n=4.

FIG. 4 shows the number of cells at Day 5 of culture when KSR at each concentration was added. n=4.

FIG. 5 specifies components of KSR disclosed in Non-Patent Literature 2.

FIG. 6 shows how addition of different factors (AlbuMAX™ II, BSA, insulin, and transferrin) affected the formation of aggregates. %=weight (w)/volume (v).

FIG. 7 indicates the percentage of human iPS cells expressing OCT 4 when subjected to adherent culture or suspension culture (containing aggregates).

FIG. 8 is micrographs obtained when a suspension containing human iPS cells was used for suspension culture while lipid-free bovine serum albumin and different lipids were added.

FIG. 9 is micrographs obtained when a suspension containing human iPS cells was used for suspension culture while lipid-free bovine serum albumin and LPA (lysophosphatidic acid) at each concentration were added. φ represents the average (±standard deviation) of the size of cell aggregates.

FIG. 10 is micrographs obtained when a suspension containing human iPS cells was used for suspension culture while lipid-free bovine serum albumin and S1P (sphingosine-1-phosphoric acid) at each concentration were added. φ represents the average (±standard deviation) of the size of cell aggregates.

FIG. 11 is micrographs obtained when a suspension containing human iPS cells was used for suspension culture while lipid-free bovine serum albumin alone, or additional LPA or S1P at each concentration was added.

FIG. 12 shows the time course of change in glucose consumption when a suspension containing human iPS cells was used for suspension culture while lipid-free bovine serum albumin alone, or additional LPA or S1P at each concentration was added. n=3.

FIG. 13 shows a total cell count at Day 5 of culture when a suspension containing human iPS cells was used for suspension culture while lipid-free bovine serum albumin alone, or additional LPA or S1P at each concentration was added. n=3.

FIG. 14 shows the percentage of human iPS cells positive for undifferentiation markers (OCT4 and SOX2) when the cells were subjected to adherent culture or suspension culture using a cell suspension containing 1.0 μg/mL of LPA or S1P.

FIG. 15 is micrographs obtained when the cells were cultured in suspension at each culture scale (4-mL scale, 300-mL scale, and 1.6-L scale) 1 day after seeding (Day 1) and 5 or 6 days after seeding (Day 5 or Day 6).

FIG. 16 shows the time course of change in cell density when the cells were cultured in suspension at each culture scale (4-mL scale, 300-mL scale, and 1.6-L scale).

FIG. 17 shows the percentage of cells positive for undifferentiation markers (OCT4 and SOX2) when the cells were subjected to adherent culture or suspension culture at each culture scale (4-mL scale, 300-mL scale, and 1.6-L scale).

### DESCRIPTION OF EMBODIMENTS

Hereinafter, one or more embodiments of the present invention will be described in detail.

### <1. Cells>

Aggregate-forming cells, which are cultured by the method of one or more embodiments of the present invention, have no particular limitation as long as they are adherent (adherent cells). Examples of the cells may include: animal-derived cells; for example mammalian-derived cells; biological tissue-derived cells and cells derived from the biological tissue-derived cells; epithelial tissue-derived cells and cells derived from the epithelial tissue-derived cells, connective tissue-derived cells and cells derived from the connective tissue-derived cells, muscular tissue-derived cells and cells derived from the muscular tissue-derived cells, or nervous tissue-derived cells and cells derived from the nervous tissue-derived cells; animal-derived stem cells and cells differentiated from the animal-derived stem cells; animal-derived pluripotent stem cells and cells differentiated from the animal-derived pluripotent stem cells; mammalian-derived pluripotent stem cells and cells differentiated from the mammalian-derived pluripotent stem cells; and human-derived pluripotent stem cells and cells differentiated from the human-derived pluripotent stem cells.

As used herein, the term “pluripotent stem cells” refers to cells that are pluripotent (multipotent) cells which can differentiate into all types of cells constituting a living body and that can continue proliferating infinitely while maintaining their pluripotent state during in vitro culture under suitable conditions. Specific examples of the pluripotent stem cells include, but are not limited to, embryonic stem cells (ES cells), EG cells, which are pluripotent stem cells derived from fetal primordial germ cells, (Shamblott M. J. et al., Proc. Natl. Acad. Sci. USA. (1998) 95, p. 13726-13731), GS cells, which are testis-derived pluripotent stem cells, (Conrad S., Nature (2008) 456, p. 344-349), and iPS cells (induced pluripotent stem cells), which are somatic cell-derived induced pluripotent stem cells. Regarding the pluripotent stem cells used in one or more embodiments of the present invention, for example, may be ES cells or iPS cells. ES cells are cultured cells derived from undifferentiated cells collected from an inner cell mass present inside an early embryo called a blastocyst. iPS cells are cultured cells produced by introducing reprogramming factors into a somatic cell, so that the somatic cell is reprogrammed into an undifferentiated state and is given pluripotency. Examples of the reprogramming factors that can be used include OCT3/4, KLF4, SOX2, and c-Myc (Yu J, et al. Science. 2007; 318:1917-20). For example, OCT3/4, SOX2, LIN28, and Nanog may be used (Takahashi K, et al. Cell. 2007; 131:861-72). Examples of how to introduce these factors into a cell include, but are not particularly limited to, a plasmid-mediated gene transfer, synthetic RNA introduction, and a direct injection of a protein(s). In addition, it may be possible to use iPS cells that are created using, for example, microRNA, RNA, and/or a low-molecular-weight compound. As the pluripotent stem cells (including the ES cells, iPS cells, etc.), commercially available products or cells obtained from a third party may be used or freshly prepared ones may be used. Examples of iPS cell lines that can be used include 253G1, 201B6, 201B7, 409B2, 454E2, HiPS-RIKEN-1A, HiPS-RIKEN-2A, HiPS-RIKEN-12A, Nips-B2, TkDN4-M, TkDA3-1, TkDA3-2, TkDA3-4, TkDA3-5, TkDA3-9, TkDA3-20, hiPSC 38-2, MSC-iPSC1, and BJ-iPSC1. Examples of ES cell lines that can be used include KhES-1, KhES-2, KhES-3, KhES-4, KhES-5, SEES1, SEES2, SEES3, HUES8, CyT49, H1, H9, and HS-181. Also, freshly prepared clinical-grade iPS or ES cells may be used. Examples of the origin of cells when iPS cells are created include, but are not particularly limited to, fibroblasts and lymphocytes.

Cells used in one or more embodiments of the present invention may be originated from any animal Examples of the origin may include: mammals such as rodents (e.g., a mouse, rat, hamster), primates (e.g., a human, gorilla, chimpanzee), and domestic animals and pets (e.g., a dog, cat, rabbit, cow, horse, sheep, goat).

In one or more embodiments of the present invention, cells isolated after undergoing adherent or suspension culture may be used. Here, the term “isolated cells” means cells obtained by detaching and dispersing a cell population composed of a plurality of cells adhering to one another. The isolation involves the step of detaching and dispersing cells adhering to, for example, cultureware and/or a culture support or a cell population, in which cells adhere to one another, to give single cells. The cell population to be isolated may be in suspension in a liquid culture medium. Examples of the isolation procedure may include, but are not particularly limited to, a procedure using a detachment agent (e.g., a cell detachment enzyme such as trypsin or collagenase), a chelating agent (e.g., EDTA (ethylene diamine tetraacetic acid)), or a mixture of the detachment agent and the chelating agent. Examples of the detachment agent include, but are not particularly limited to, trypsin, Accutase (a registered trade mark), TrypLE™ Express Enzyme (Life Technologies Japan Ltd.), TrypLE™ Select Enzyme (Life Technologies Japan Ltd.), “Dispase” (a registered trade mark), and collagenase. The cells that have been isolated, frozen, and stored after the isolation procedure may be used in one or more embodiments of the present invention.

### <2. Cell Aggregates>

As used herein, a cell aggregate refers to what is called a spheroid that is a clustered body formed while a plurality of cells aggregate three-dimensionally. Cell aggregates prepared in accordance with one or more embodiments of the present invention typically have a substantially spherical shape.

As used herein, aggregate-forming cells have no particular limitation as long as they contain at least one type of the above adherent cells. For example, cells expressing a pluripotent stem cell marker are included in the cell aggregate composed of pluripotent stem cells (e.g., human pluripotent stem cells, human embryonic stem cells). Examples of the pluripotent stem cell maker include alkaline phosphatase, NANOG, OCT4, SOX2, TRA-1-60, c-Myc, KLF4, LIN28, SSEA-4, and SSEA-1. When the aggregate-forming cells are the pluripotent stem cells, the percentage of cells expressing the pluripotent stem cell marker may be 80% or higher, 90% or higher, 91% or higher, 92% or higher, 93% or higher, 94% or higher, 95% or higher, 96% or higher, 97% or higher, 98% or higher, 99% or higher, or 100% or less.

The size of cell aggregates as produced by the method of one or more embodiments of the present invention has no particular limitation. When observed under a microscope, the upper limit of the size of the widest portion on a micrograph may be 1000 μm, 900 μm, 800 μm, 700 μm, 600 μm, 500 μm, 400 μm, or 300 μm. The lower limit may be 50 μm, 60 μm, 70 μm, 80 μm, 90 μm, or 100 μm. The cell aggregates with such a size range have a preferable cell growth environment because oxygen and nutritional components are easily supplied to their inner cells.

When measured by weight, 10% or higher, 20% or higher, 30% or higher, 40% or higher, 50% or higher, 60% or higher, 70% or higher, 80% or higher, or 90% or higher of the cell aggregates constituting a cell aggregate population prepared by the method of one or more embodiments of the present invention may have a size within the above range.

### <3. Liquid Culture Medium>

Regarding a liquid culture medium used in one or more embodiments of the present invention, any of liquid culture media for culturing an animal cell may be used as a basal medium. The liquid culture medium of interest may be prepared by appropriately adding, if necessary, another component.

Examples of the basal medium that can be used include, but are not particularly limited to, BME medium, BGJb medium, CMRL1066 medium, Glasgow MEM medium, Improved MEM Zinc Option medium, IMDM medium (Iscove's Modified Dulbecco's Medium), Medium 199 medium, Eagle MEM medium, (MEM medium, DMEM medium (Dulbecco's Modified Eagle's Medium), Ham's F10 medium, Ham's F12 medium, RPMI 1640 medium, Fischer's medium, and a mixed medium thereof (e.g., DMEM/F12 medium (Dulbecco's Modified Eagle's Medium/Nutrient Mixture F-12 Ham)). The DMEM/F12 medium may be used, e.g., by mixing DMEM medium and Ham's F12 medium in a weight ratio of from 60/40 to 40/60, from 55/45 to 45/55, or 50/50.

The liquid culture medium used in one or more embodiments of the present invention may be a medium containing no serum, namely a serum-free medium.

The liquid culture medium used in one or more embodiments of the present invention may contain at least one selected from L-ascorbic acid, insulin, transferrin, selenium, and sodium bicarbonate, or may contain all of them. The L-ascorbic acid, insulin, transferrin, selenium, and sodium bicarbonate may be added to the medium in the form of, for example, a solution, derivative, salt, or mixed reagent. For example, L-ascorbic acid may be added to the medium in the form of a derivative such as magnesium-ascorbyl-2-phosphate. Selenium may be added to the medium in the form of a selenite (e.g., sodium selenite). The insulin and transferrin may be natural ones isolated from a tissue or serum of an animal (e.g., a human, mouse, rat, cow, horse, goat). They may be genetically engineered recombinant proteins. The insulin, transferrin, and selenium may be added to the medium in the form of a reagent ITS (insulin-transferrin-selenium). The ITS is a cell growth-promoting additive containing insulin, transferrin, and sodium selenite.

A commercially available culture medium containing at least one selected from L-ascorbic acid, insulin, transferrin, selenium, and sodium bicarbonate may be used as a liquid culture medium of one or more embodiments of the present invention. Examples of a commercially available culture medium supplemented with insulin and transferrin may include CHO-S-SFM II (Life Technologies Japan Ltd.), Hybridoma-SFM (Life Technologies Japan Ltd.), eRDF Dry Powdered Media (Life Technologies Japan Ltd.), UltraCULTURE™ (BioWhittaker, Inc.), UltraDOMA™ (BioWhittaker, Inc.), UltraCHO™ (BioWhittaker, Inc.), and UltraMDCK™ (BioWhittaker, Inc.). For, example, STEMPRO (a registered trademark), hESC SFM (Life Technologies Japan Ltd.), mTeSR1 (Veritas, Ltd.), or TeSR2 (Veritas, Ltd.) may be used. In addition, a liquid culture medium used for culturing human iPS cells and/or human ES cells may be used.

The liquid culture medium used in one or more embodiments of the present invention may contain at least one growth factor. The liquid culture medium may contain at least one growth factor, which is not limited to the following, selected from the group consisting of FGF2 (basic fibroblast growth factor-2), TGF-β1 (transforming growth factor-β1), Activin A, IGF-1, MCP-1, IL-6, PAI, PEDF, IGFBP-2, LIF, and IGFBP-7.

A serum-free medium may be used as the liquid culture medium in one or more embodiments of the present invention, which contains, in addition to albumin and/or the below-described lipid, components: L-ascorbic acid, insulin, transferrin, selenium, and sodium bicarbonate as well as at least one growth factor. A serum-free DMEM/F12 medium may be used, which contains L-ascorbic acid, insulin, transferrin, selenium, and sodium bicarbonate as well as at least one growth factor (e.g., FGF2 and TGF-β1). Examples of such a medium that may be used include Essential 8™ medium (Life Technologies Japan Ltd.) supplemented with albumin and/or the below-described lipid. The Essential 8™ medium may be prepared by mixing DMEM/F-12 (HAM) (1:1), which is a DMEM/F12 medium marketed by Life Technologies Japan Ltd., and Essential 8™ supplement (containing L-ascorbic acid, insulin, transferrin, selenium, sodium bicarbonate, FGF2, and TGF-β1).

The liquid culture medium used in one or more embodiments of the present invention may contain components such as fatty acids or lipids, amino acids (e.g., non-essential amino acids), vitamins, cytokines, antioxidants, 2-mercaptoethanol, pyruvic acid, buffers, inorganic salts, antibiotics, and kinase inhibitors.

Examples of the antibiotics that may be used include penicillin, streptomycin, and amphotericin B.

Examples of the kinase inhibitors that may be added include ROCK inhibitors.

The ROCK inhibitors are defined as a substance that inhibits the kinase activity of Rho kinase (ROCK, a Rho-associated protein kinase). Examples include: Y-27632 (4-[(1R)-1-aminoethyl]-N-pyridine-4-ylcyclohexane-1-carboxamide) or a dihydrochloride thereof (see, for example, Ishizaki et al., Mol. Pharmacol. 57, 976-983 (2000); Narumiya et al., Methods Enzymol. 325,273-284 (2000)); Fasudil/HA1077 (1-(5-isoquinoline sulfonyl)homopiperazine) or a dihydrochloride thereof (see, for example, Uenata et al., Nature 389: 990-994 (1997)); H-1152((S)-(+)-2-methyl-1-[(4-methyl-5-isoquinolinyl) sulfonyl]-hexahydro-1H-1,4-diazepine) or a dihydrochloride thereof (see, for example, Sasaki et al., Pharmacol. Ther. 93: 225-232 (2002)); Wf-536((+)-(R)-4-(1-aminoethyl)-N-(4-pyridyl)benzamide monohydrochloride) (see, for example, Nakajima et al., Cancer Chemother. Pharmacol. 52(4): 319-324 (2003)) and a derivative thereof; and antisense nucleic acid against ROCK, RNA interference-inducing nucleic acid (e.g., siRNA), a dominant negative mutant, and vectors expressing these molecules. In addition, because other low-molecular-weight compounds have been known as the ROCK inhibitors, such compounds or derivatives thereof may be used in accordance with one or more embodiments of the present invention (see, for example, US Patent Application Publication Nos. 20050209261, 20050192304, 20040014755, 20040002508, 20040002507, 20030125344, and 20030087919, and WO2003/062227, WO2003/059913, WO2003/062225, WO2002/076976, and WO2004/039796). In one or more embodiments of the present invention, at least one kind of the ROCK inhibitor may be used.

The ROCK inhibitor used in one or more embodiments of the present invention may be Y-27632, which is a compound represented by the following formula I or a salt (e.g., a dihydrochloride) thereof. Y-27632 may be added as the form of a hydrate.

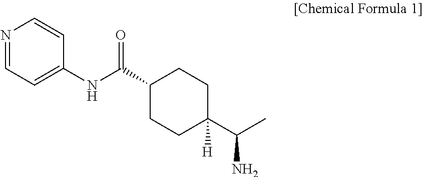 [SMILES: C[C@@H](N)[C@H]1CC[C@H](C(=O)Nc2ccncc2)CC1]

The concentration of Y-27632 in a liquid culture medium is not limited and is, for example, from 80 to 120 nM (e.g., 100 nM), from 400 to 600 nM (e.g., 500 nM), from 600 to 900 nM (e.g., 750 nM), from 0.8 to 1.2 μM (e.g., 1 μM), from 1.6 to 2.4 μM (e.g., 2 μM), from 2.4 to 3.6 μM (e.g., 3 μM), from 3.2 to 4.8 μM (e.g., 4 μM), from 4 to 6 μM (e.g., 5 μM), from 4.8 to 7.2 μM (e.g., 6 μM), μM (e.g., 7 μM), from 6.4 to 9.6 μM (e.g., 8 μM), from 7.2 to 10.8 μM (e.g., 9 μM), from 8 to 12 μM (e.g., 10 μM), from 12 to 18 μM (e.g., 15 μM), from 16 to 24 μM (e.g., 20 μM), from 20 to 30 μM (e.g., 25 μM), from 24 to 36 μM (e.g., 30 μM), from 32 to 48 μM (e.g., 40 μM), from 40 to 60 μM (e.g., 50 μM), or from 8 to 12 μM (e.g., 10 μM).

### <4. Lipids>

A lipid according to one or more embodiments of the present invention may have an ability to bind to albumin. In this section <4. Lipids>, unless otherwise indicated, the “lipid” refers to a lipid having an ability to bind to albumin Specific examples of the lipid include several forms of lipids: lipids that form a complex with a protein (e.g., serum albumin); lipids isolated from serum albumin; and lipids that are produced in microorganisms or produced through chemical synthesis and that have an ability to bind to albumin. Note that the “ability to bind to albumin” refers to a characteristic in which the lipid can form a complex with albumin by means of chemical and/or physical interaction.

Examples of animals from which the lipid is derived may include: mammals such as rodents (e.g., a mouse, rat, hamster), primates (e.g., a human, gorilla, chimpanzee), and domestic animals and pets (e.g., a dog, cat, rabbit, cow, horse, sheep, goat, pig). The lipid may be derived from the same organism species as of cells to be cultured. In addition, the lipid may be artificially synthesized.

The lipid may be added, as a serum containing the lipid, to a liquid culture medium. In addition, the lipid may be added, as serum albumin containing the lipid (lipid-containing serum albumin), to a liquid culture medium. As the lipid-containing serum albumin, it is possible to use a lipid-protein (of serum albumin) complex. Among various types of the lipid-containing serum albumin, those having an increased content of lipid may be used. Examples of the lipid-containing serum albumin include commercially available AlbuMAX™. Specific examples are AlbuMAX™ I or AlbuMAX™ II (both come from Life Technologies Japan Ltd.).

The lipid had no particular limitation as long as the lipid is derived from serum or can bind to serum albumin. Also, the lipid may vary depending on a source organism. Thus, the lipid may be a free fatty acid, phospholipid (e.g., a glycerophospholipid, sphingophospholipid, lysophosphatidylcholine), neutral fat, or cholesterol. The lipid, for example, contains at least one, selected from the group consisting of lysophosphatidylcholine, triacylglyceride, phosphatidylcholine, phosphatidic acid, cholesterol, and sphingomyelin. The lipid used in one or more embodiments of the present invention may contain, as a total lipid weight basis, 20 to 80 wt % (or 40 to 70 wt %) of a free fatty acid, 5 to 50 wt % (or 10 to 30 wt %) of lysophosphatidylcholine, 5 to 45 wt % (or 10 to 30 wt %) of a triacylglyceride, and 2 to 25 wt % (or 5 to 15 wt %) of phosphatidylcholine, at least. The lipid may further contain at least one selected from the group consisting of 1 to 10 wt % (or 1 to 6 wt %) of phosphatidic acid, 0.1 to 3 wt % (or 0.5 to 2 wt %) of cholesterol, and 0.1 to 3 wt % (or 0.5 to 2 wt %) of sphingomyelin. The lipid-containing serum albumin containing such a composition of lipid may be used for the method of one or more embodiments of the present invention. Note that according to Non-Patent Literature 2, AlbuMAX™ II contains, as a total lipid weight basis, about 54 wt % of a free fatty acid, about 17 wt % of lysophosphatidylcholine, about 15 wt % of a triacylglyceride, about 8 wt % of phosphatidylcholine, about 3 wt % of phosphatidic acid, about 1 wt % of cholesterol, and about 1 wt % of sphingomyelin.

The lipids isolated from serum albumin may be used in one or more embodiments of the present invention. Examples of a method for isolating the lipids from the serum albumin include, but are not particularly limited to, a method comprising subjecting a lipid-containing serum albumin complex to protease (e.g. trypsin) treatment to recover the lipids. The isolated lipids may be used in one or more embodiments of the present invention while the composition of the lipids contained in the serum albumin is kept substantially the same. Alternatively, some of the lipids may be enriched and used.

The lipids may be included in a serum replacement commercially available. In this case, the lipids may be added, to a liquid culture medium, as one component included in the serum replacement and then used. The serum replacement is a reagent used as a serum (e.g., FBS) replacement so as to maintain an undifferentiated state of ES cells or iPS cells and culture them. Examples of the serum replacement include KNOCKOUT™ SR (KnockOut™ Serum Replacement (KSR); Life Technologies Japan Ltd.), StemSure (a registered trademark) Serum Replacement (SSR; Wako Pure Chemical Industries, Ltd.), and N2 supplement (Wako Pure Chemical Industries, Ltd.). The above KSR contains AlbuMAX™, specifically, AlbuMAX™ I or AlbuMAX™ II.

Generally speaking, the concentration of albumin included in serum is about 50 mg/ml. It reasonably assumes that the KSR contains a similar concentration of serum albumin According to Non-Patent Literature 2, the serum albumin included in the KSR is AlbuMAX™. In addition, Non-Patent Literature 2 discloses that the amount of lipids included in 100 mg of AlbuMAX™ is 0.65 mg. Thus, a liquid culture medium containing a final concentration of 1% (v/v) KSR, for example, contains 0.5 mg/ml of AlbuMAX™, and, accordingly, the concentration of lipids included is 0.00325 mg/ml. For example, a liquid culture medium containing a final concentration of 2 mg/ml AlbuMAX™ contains 0.013 mg/mL of lipids. Hence, the content of lipids in a liquid culture medium used for suspension culture according to one or more embodiments of the present invention has no particular limitation and may be from 0.00325 to 0.065 mg/ml, from 0.00325 to 0.0325 mg/ml, from 0.00325 to 0.01625 mg/ml, or from 0.00325 to 0.013 mg/ml. The concentration of lipids may be determined by appropriate analysis protocols (e.g., analysis protocols using liquid chromatography, gas chromatography, and other means).

The lipid may be present in a liquid culture medium under conditions in which the lipid is free of albumin. A separately prepared lipid and albumin may be added to a liquid culture medium. Examples of the origin of albumin added include, but are not particularly limited to, human or bovine albumin That is, a method according to one or more embodiments of the present invention may comprise a step of adding an albumin-free lipid to prepare a liquid culture medium. In addition, lipid-free serum albumin may be employed. It may also be possible to use albumin as prepared by expressing the albumin in E. coli or animal cells using a gene recombinant technology. Proteins or additives, which replace albumin, for example, amphiphilic substances (e.g., a surfactant) may also be added.

### <5. Lysophospholipid>

A lysophospholipid is used as a lipid according to one or more embodiments of the present invention.

The lysophospholipid is a general term for a phospholipid having an aliphatic group (e.g., a medium or long chain aliphatic group). The lysophospholipid may have a glycerol or sphingosine backbone. Examples of the lysophospholipid include lysophosphatidic acid (LPA), sphingosine-1-phosphoric acid (S1P), lysophosphatidylcholine (LPC), lysophosphatidylserine (LPS), lysophosphatidylinositol (LPI), lysophosphatidylglycerol (LPG), lysophosphatidylthreonine (LPT), and lysophosphatidyl ethanolamine (LPE). It may be a mixture containing a plurality of lysophospholipids. These lysophospholipids may be in any form (e.g., a salt). The lysophospholipid may be a lysophospholipid other than LPC. LPA and S1P may be used, which are a lysophospholipid having a non-substituted phosphate group as a polar head group. When the lysophospholipid contains an acyl group, the number of carbons and the degree of unsaturation of the acyl group have no particular limitation. The number of carbons and the degree of unsaturation of the acyl group may depend on a source organism. Usually, the acyl group has 16 to 24 carbons and the degree of unsaturation ranges from 0 to 6. The number of carbons:the degree of unsaturation in the acyl group may be 16:0, 16:1, 18:0, 18:1, 18:2, 18:3, 20:0, 20:1, 20:2, 20:3, 20:4, 20:5, 22:0, 22:1, 22:2, 22:3, 22:4, 22:5, or, 22:6. In addition, the lysophospholipid with a glycerol backbone may be either 1-acyl lysophospholipid or 2-acyl lysophospholipid.

The source organism of the lysophospholipid may be selected from those of a similar source organism range described in the section <4. Lipids>. In addition, the lysophospholipid may be artificially prepared.

The lysophospholipid may be added to a liquid culture medium as a lysophospholipid-containing composition. Examples of the composition include a mixture of a lysophospholipid and a protein.

The lysophospholipid that is not in complex with a protein may be used in a culture. A lysophospholipid not in the form of a mixture with a protein, for example a lysophospholipid in a substantially purified form, may be used as a material for preparation of a liquid culture medium. In this case, it is easy to adjust the additive amount of the lysophospholipid to a preferable range. Such a lysophospholipid may be isolated from a source organism or may be artificially prepared.

At the start of suspension culture, the concentration of the lysophospholipid in a liquid culture medium may be adjusted appropriately. For example, the concentration may be 0.00128 μg/mL or more, 0.0064 μg/mL or more, more than 0.0064 μg/mL, 0.032 μg/mL or more, 0.064 μg/mL or more, 0.16 μg/mL or more, 0.2 μg/mL or more, 0.3 μg/mL or more, 0.4 μg/mL or more, or 0.5 μg/mL or more. In this case, aggregation of cells can be moderately suppressed to form cell aggregates with a substantially uniform size. For example, the concentration may be 1000 μg/mL or less, 200 μg/mL or less, 150 μg/mL or less, 100 μg/mL or less, 90 μg/mL or less, 80 μg/mL or less, 70 μg/mL or less, 60 μg/mL or less, 50 μg/mL or less, 40 μg/mL or less, 30 μg/mL or less, 20 μg/mL or less, 10 μg/mL or less, 9 μg/mL or less, 8 μg/mL or less, 7 μg/mL or less, 6 μg/mL or less, 5 μg/mL or less, 4 μg/mL or less, 3 μg/mL or less, 2 μg/mL or less, 1 μg/mL or less, 0.9 μg/mL or less, 0.8 μg/mL or less, 0.7 μg/mL or less, or 0.6 μg/mL or less. In the above cases, spherical cell aggregates with the above-mentioned suitable size may be formed. A plurality of kinds of lysophospholipids may be added, as components, to a liquid culture medium. In this case, the concentration of each lysophospholipid may be set to within the above range. The total concentration of the lysophospholipids may be set to within the above range. Note that the amount of the above lysophospholipid refers to the amount of a lysophospholipid added when a liquid culture medium is prepared (provided that when a lysophospholipid is generated through an enzymatic reaction in a liquid culture medium as described below, the amount of interest includes the amount of a lysophospholipid generated by this reaction). The amount of interest does not include the amount of lysophospholipids produced by cultured cells. The concentration of the lysophospholipid may be determined by appropriate analysis protocols (e.g., analysis protocols using liquid chromatography, gas chromatography, and other means). The concentration by weight of the lysophospholipid may be determined in terms of 2S-amino-1-(dihydrogen phosphate)-4E-octadecene-1,3R-diol (with a molecular weight of 379.5) is within the above range.

The lysophospholipid may be lysophosphatidic acid. In this case, at the start of suspension culture, the concentration of the lysophosphatidic acid in a liquid culture medium may be adjusted appropriately. For example, the concentration is 0.00128 μg/mL or more or 0.00279 μM or more, 0.0064 μg/mL or more or 0.0140 μM or more, more than 0.0064 μg/mL or more than 0.0140 μM, 0.032 μg/mL or more or 0.0698 μM or more, 0.064 μg/mL or more or 0.140 μM or more, 0.16 μg/mL or more or 0.349 μM or more, 0.2 μg/mL or more or 0.436 μM or more, 0.3 μg/mL or more or 0.654 μM or more, 0.4 μg/mL or more or 0.872 μM or more, or 0.5 μg/mL or more or 1.09 μM or more. In the above cases, aggregation of cells may be moderately suppressed to form cell aggregates with a substantially uniform size. For example, the concentration may be 1000 μg/mL or less or 2180 μM or less, 200 μg/mL or less or 436 μM or less, 150 μg/mL or less or 327 μM or less, 100 μg/mL or less or 218 μM or less, 90 μg/mL or less or 196 μM or less, 80 μg/mL or less or 174 μM or less, 70 μg/mL or less or 153 μM or less, 60 μg/mL or less or 131 μM or less, 50 μg/mL or less or 109 μM or less, 40 μg/mL or less or 87.2 μM or less, 30 μg/mL or less or 65.4 μM or less, 20 μg/mL or less or 43.6 μM or less, 10 μg/mL or less or 21.8 μM or less, 9 μg/mL or less or 19.6 μM or less, 8 μg/mL or less or 17.4 μM or less, 7 μg/mL or less or 15.3 μM or less, 6 μg/mL or less or 13.1 μM or less, 5 μg/mL or less or 10.9 μM or less, 4 μg/mL or less or 8.72 μM or less, 3 μg/mL or less or 6.54 μM or less, 2 μg/mL or less or 4.36 μM or less, 1 μg/mL or less or 2.18 μM or less, 0.9 μg/mL or less or 1.96 μM or less, 0.8 μg/mL or less or 1.74 μM or less, 0.7 μg/mL or less or 1.53 μM or less, or 0.6 μg/mL or less or 1.31 μM or less. In the above cases, spherical cell aggregates with the above-mentioned suitable size may be formed. Note that the amount of the above lysophosphatidic acid refers to the amount of a lysophosphatidic acid added when a liquid culture medium is prepared (provided that when a lysophosphatidic acid is generated through an enzymatic reaction in a liquid culture medium as described below, the amount of interest includes the amount of a lysophosphatidic acid generated by this reaction). The amount of interest does not include the amount of lysophosphatidic acid produced by cultured cells. The concentration by weight of the above lysophosphatidic acid refers to the concentration by weight in terms of a sodium salt thereof (1-O-9Z-octadecenoyl-sn-glyceryl-3-phosphoric acid, sodium salt; with a molecular weight of 458.5).

The lysophospholipid may be sphingosine-1-phosphoric acid. In this case, at the start of suspension culture, the concentration of the sphingosine-1-phosphoric acid in a liquid culture medium may be adjusted appropriately. For example, the concentration may be 0.00128 μg/mL or more or 0.00337 μM or more, 0.0064 μg/mL or more or 0.0169 μM or more, more than 0.0064 μg/mL or more than 0.0169 μM, 0.032 μg/mL or more or 0.0843 μM or more, 0.064 μg/mL or more or 0.169 μM or more, 0.16 μg/mL or more or 0.422 μM or more, 0.2 μg/mL or more or 0.527 μM or more, 0.3 μg/mL or more or 0.791 μM or more, 0.4 μg/mL or more or 1.05 μM or more, or 0.5 μg/mL or more or 1.32 μM or more. In the above cases, aggregation of cells can be moderately suppressed to form cell aggregates with a substantially uniform size. For example, the concentration may be 1000 μg/mL or less or 2640 μM or less, 200 μg/mL or less or 527 μM or less, 150 μg/mL or less or 395 μM or less, 100 μg/mL or less or 264 μM or less, 90 μg/mL or less or 237 μM or less, 80 μg/mL or less or 211 μM or less, 70 μg/mL or less or 184 μM or less, 60 μg/mL or less or 158 μM or less, 50 μg/mL or less or 132 μM or less, 40 μg/mL or less or 105 μM or less, 30 μg/mL or less or 79.1 μM or less, 20 μg/mL or less or 52.7 μM or less, 10 μg/mL or less or 26.4 μM or less, 9 μg/mL or less or 23.7 μM or less, 8 μg/mL or less or 21.1 μM or less, 7 μg/mL or less or 18.4 μM or less, 6 μg/mL or less or 15.8 μM or less, 5 μg/mL or less or 13.2 μM or less, 4 μg/mL or less or 10.5 μM or less, 3 μg/mL or less or 7.91 μM or less, 2 μg/mL or less or 5.27 μM or less, 1 μg/mL or less or 2.64 μM or less, 0.9 μg/mL or less or 2.37 μM or less, 0.8 μg/mL or less or 2.11 μM or less, 0.7 μg/mL or less or 1.84 μM or less, or 0.6 μg/mL or less or 1.58 μM or less. In the above cases, spherical cell aggregates with the above-mentioned suitable size may be formed. Note that the amount of the above sphingosine-1-phosphoric acid refers to the amount of a sphingosine-1-phosphoric acid added when a liquid culture medium is prepared (provided that when sphingosine-1-phosphoric acid is generated through an enzymatic reaction in a liquid culture medium as described below, the amount of interest includes the amount of sphingosine-1-phosphoric acid generated by the above reaction). The amount of interest does not include the amount of sphingosine-1-phosphoric acid produced by cultured cells. The concentration by weight of the sphingosine-1-phosphoric acid may refer to the concentration by weight determined in terms of a free form thereof (2S-amino-1-(dihydrogen phosphate)-4E-octadecene-1,3R-diol; with a molecular weight of 379.5).

A plurality of the lysophospholipids may be mixed and used. Regarding the mixed ratio, examples of the ratio by weight of LPA to S1P include, but are not particularly limited to, 1:1 to 80000 or 1 to 80000:1. Specific examples of the ratio that can be used include 1:0.5 to 1.5 (e.g., 1:1), 1:2.5 to 7.5 (e.g., 1:5), 1:13 to 38 (e.g., 1:25), 1:63 to 190 (e.g., 1:125), 1:78 to 230 (e.g., 1:156.25), 1:310 to 940 (e.g., 1:625), 1:1600 to 4700 (e.g., 1:3125), 1:7800 to 23000 (e.g., 1:15625), 1:39000 to 120000 (e.g., 1:78125), 2.5 to 7.5:1 (e.g., 5:1), 13 to 38:1 (e.g., 25:1), 63 to 190:1 (e.g., 125:1), 78 to 230:1 (e.g., 156.25:1), 78 to 230:5 (e.g., 156.25:5), 78 to 230:25 (e.g., 156.25:25), 78 to 230:125 (e.g., 156.25:125), 310 to 940:1 (e.g., 625:1), 310 to 940:156.25 (e.g., 625:156.25), 1600 to 4700:1 (e.g., 3125:1), 1600 to 4700:156.25 (e.g., 3125:156.25), 7800 to 23000:1 (e.g., 15625:1), 7800 to 23000:156.25 (e.g., 15625:156.25), 39000 to 120000:1 (e.g., 78125:1), and 39000 to 120000:156.25 (e.g., 78125:156.25). LPA may be determined in terms of a sodium salt thereof (1-O-9Z-octadecenoyl-sn-glyceryl-3-phosphoric acid, sodium salt; with a molecular weight of 458.5) and S1P may be determined in terms of a free form thereof (2S-amino-1-(dihydrogen phosphate)-4E-octadecene-1,3R-diol; with a molecular weight of 379.5) to calculate the weight ratio.

Lysophospholipids including lysophosphatidylcholine (LPC), lysophosphatidylserine (LPS), lysophosphatidylinositol (LPI), lysophosphatidylglycerol (LPG), lysophosphatidylthreonine (LPT), or lysophosphatidyl ethanolamine (LPE) may be used in the above concentration or mixed ratio in addition to lysophosphatidic acid (LPA) and sphingosine-1-phosphoric acid (S1P).

The method of one or more embodiments of the present invention may comprise a step of carrying out an enzymatic reaction to produce from a lipid the above lysophospholipid. Examples of the enzymatic reaction may include a hydrolase reaction and a kinase reaction.

Examples of the hydrolase reaction include hydrolysis catalyzed by a hydrolase using the above phospholipid as a substrate. Examples of the hydrolase used in one or more embodiments of the present invention include, but are not particularly limited to, phospholipases and lysophospholipases. Examples of the phospholipases and lysophospholipases include, but are not particularly limited to, phospholipase A1, phospholipase A2, and lysophospholipase D (Autotaxin). Phospholipase A1 hydrolyzes an ester bond at sn-1 position of a glycerophospholipid. Phospholipase A2 hydrolyzes an ester bond at sn-2 position of a glycerophospholipid. Autotaxin is a hydrolase that hydrolyzes a phosphoester bond between a phosphate group and a substituent of a phospholipid or lysophospholipid to generate an unsubstituted phosphate group as a polar head group. Autotaxin can hydrolyze LPC to produce LPA and choline. Phospholipase A1 or phospholipase A2 and Autotaxin hydrolyze a glycerophospholipid as a substrate to produce hydrolysates (e.g., LPA), which may be used as a lysophospholipid in one or more embodiments of the present invention. For example, LPC, LPS, LPI, LPG, LPT, or LPE, is subject to the above Autotaxin treatment to give hydrolysates (e.g., LPA) that may be used in one or more embodiments of the present invention.

In one or more embodiments of the present invention, S1P that is prepared from the above sphingo lipid may be used as a lysophospholipid. The preparation method has no particular limitation. For example, a sphingosine is phosphorylated in the presence of a kinase such as a sphingokinase to give a phosphorylated product (e.g., S1P), which may be used as a lysophospholipid.

The method of one or more embodiments of the present invention may further comprise a step of adding the above lysophospholipid to prepare a liquid culture medium. At this time, the above lysophospholipid may be added in a form not in complex with a protein because it is easy to control the additive amount of the lysophospholipid.

In one or more embodiments of the present invention, a cell aggregation inhibition kit comprising the lysophospholipid (e.g., either LPA or S1P), the hydrolysate of the lipid, a mixture of the lipid and the hydrolase, a mixture of the sphingo lipid and the kinase, or a liquid culture medium containing any of these materials, may be used.

### <6. Cell Aggregation Inhibitor>

A cell aggregation inhibitor according to one or more embodiments of the present invention contains the above lipid and may appropriately inhibit aggregation of cells in a suspension culture system to form cell aggregates with a substantially uniform size.

In one or more embodiments of the present invention, the cell aggregation inhibitor may be in any form. The cell aggregation inhibitor may be the above lipid itself or may be a composition produced by combining the above lipid and another component. The form of the composition is not particularly limited. The composition may be, for example, a liquid culture medium used for suspension culture or may be an additive composition mixed when a liquid culture medium is prepared.

In one or more embodiments of the present invention, the cell aggregation inhibitor may be the liquid culture medium or a buffer (e.g., a phosphate buffer) containing the lysophospholipid(s) in the above-described concentration or ratio.

In one or more embodiments of the present invention, the cell aggregation inhibitor may be a liquid composition containing the lysophospholipid(s) in a liquid medium. The liquid composition is an additive mixed when a liquid culture medium for suspension culture is prepared. The liquid composition may be prepared such that the final concentration of the lysophospholipid in a liquid culture medium prepared is within the above described concentration. The concentration of the lysophospholipid in the liquid composition before mixed with the cells of interest has no particular limitation. The concentration of the lysophospholipid may be 1 or more, 10 or more, 100 or more, 1000 or more, or 10000 or more times the above-mentioned concentration specified as a concentration during suspension culture.

The cell aggregation inhibitor may also contain, as additives, an enzyme (e.g., a hydrolase, kinase), antibiotic, kinase inhibitor, buffer, thickener, colorant, stabilizer, surfactant, emulsifier, preservative, preserving agent, or antioxidant. The enzyme is not particularly limited and a hydrolase or a kinase, for example, may be used. The hydrolase or kinase is as described above. Examples of the antibiotics that can be used include, but are not particularly limited to, penicillin, streptomycin, and amphotericin B. Examples of the kinase inhibitor include, but are not particularly limited to, ROCK inhibitors. Examples of the ROCK inhibitors include, but are not particularly limited to, Y-27632. The cell aggregation inhibitor according to one or more embodiments of the present invention may contain a ROCK inhibitor in the above-described concentration as a final concentration in a liquid culture medium. The final concentration of the ROCK inhibitor in a liquid culture medium may be 10 μM. Examples of the buffer include a phosphate buffer, tris-hydrochloric acid buffer, and glycine buffer. Examples of the thickener include gelatin and polysaccharides. Examples of the colorant include Phenol Red. Examples of the stabilizer include albumin, dextran, methyl cellulose, and gelatin. Examples of the surfactant include cholesterol, an alkyl glycoside, alkyl polyglycoside, alkyl monoglyceride ether, glucoside, maltoside, neopentyl glycol series, polyoxyethylene glycol series, thioglucoside, thiomaltoside, peptide, saponin, phospholipid, sorbitan fatty acid ester, and fatty acid diethanolamide. Examples of the emulsifier include a glycerin fatty acid ester, sorbitan fatty acid ester, propylene glycol fatty acid ester, and sucrose fatty acid ester. Examples of the preservative include aminoethyl sulfonic acid, benzoic acid, sodium benzoate, ethanol, sodium edetate, agar, dl-camphor, citric acid, sodium citrate, salicylic acid, sodium salicylate, phenyl salicylate, dibutylhydroxy toluene, sorbic acid, potassium sorbate, nitrogen, dehydro acetic acid, sodium dehydroacetate, 2-naphthol, white soft sugar, honey, paraoxy isobutyl benzoate, paraoxy isopropyl benzoate, paraoxy ethyl benzoate, paraoxy butyl benzoate, paraoxy propyl benzoate, paraoxy methyl benzoate, 1-menthol, and eucalyptus oil. Examples of the preserving agent include benzoic acid, sodium benzoate, ethanol, sodium edetate, dried sodium sulfite, citric acid, glycerin, salicylic acid, sodium salicylate, dibutylhydroxy toluene, D-sorbitol, sorbic acid, potassium sorbate, sodium dehydroacetate, paraoxy isobutyl benzoate, paraoxy isopropyl benzoate, paraoxy ethyl benzoate, paraoxy butyl benzoate, paraoxy propyl benzoate, paraoxy methyl benzoate, propylene glycol, and phosphoric acid. Examples of the antioxidant include citric acid, citric acid derivatives, vitamin C and derivatives thereof, lycopene, vitamin A, carotenoids, vitamin B and derivatives thereof, flavonoids, polyphenols, glutathione, selenium, sodium thiosulfate, vitamin E and derivatives thereof, α-lipoic acid and derivatives thereof, pycnogenol, flavangenol, super oxide dismutase (SOD), glutathione peroxidase, glutathione-S-transferase, glutathione reductase, catalase, ascorbic acid peroxidase, and mixtures thereof.

The cell aggregation inhibitor may contain a growth factor. The cell aggregation inhibitor may contain at least one of FGF2 and TGF-β1.

### <7. Suspension Culture>

In one or more embodiments of the present invention, cells are cultured in suspension in a liquid culture medium to form cell aggregates. In one or more embodiments of the present invention, cells may be cultured in suspension under conditions in which, assuming that the prescribed lipid of one or more embodiments of the present invention is not present in the liquid culture medium, cell aggregates with more than the above-described suitable size would be formed.

Cultureware used for suspension cell culture may be a container on which cells adhere less to an inner surface thereof. Examples of such a container include plates, the surface of which is subjected to hydrophilic treatment with a biocompatible substance. Examples of the cultureware that may be used include, but are not particularly limited to, Nunclon™ Sphera (Thermo Fisher Scientific Inc.).

Examples of the shape of the cultureware include, but are not particularly limited to, a dish, flask, well, bag, and spinner flask shape.

The suspension culture may be static culture. Also, the culture may be performed under conditions in which a liquid culture medium flows. The culture may be performed under conditions in which a liquid culture medium flows. When the culture is performed under conditions in which a liquid culture medium flows, the culture may be performed under the conditions so as to promote cell aggregation. Examples of this culture include: culture under conditions in which a liquid culture medium flows such that cells are concentrated on a spot due to stress (e.g., centrifugal force, centripetal force) caused by, for example, a swirling and/or rocking flow; and culture under conditions in which a liquid culture medium flows due to a linear back and forth movement.

The rotary shaking culture (i.e., culture under shaking) is carried out such that cultureware housing cells in a liquid culture medium is subject to rotary shaking substantially along the horizontal plane in a closed path (e.g., a circle, ellipse, flat circle, flat ellipse). The speed of rotation has no particular limitation and the upper limit may be 200 rpm, 150 rpm, 120 rpm, 115 rpm, 110 rpm, 105 rpm, 100 rpm, 95 rpm, or 90 rpm. The lower limit may be 1 rpm, 10 rpm, 50 rpm, 60 rpm, 70 rpm, 80 rpm, or 90 rpm. The shaking width during rotary shaking culture has no particular limitation and the lower limit may be, for example, 1 mm, 10 mm, 20 mm, or 25 mm. The upper limit of the shaking width may be, for example, 200 nm, 100 mm, 50 mm, 30 mm, or 25 mm. The radius of rotation during rotary shaking culture has no particular limitation and may be set such that the shaking width is within the above-described range. The lower limit of the radius of rotation may be, for example, 5 mm or 10 mm. The upper limit of the radius of rotation may be, for example, 100 mm or 50 mm Setting the rotary shaking culture condition to this range may be used to prepare cell aggregates with an appropriate size easily.

The rocking culture is carried out while a liquid culture medium flows and is mixed by rocking. The rocking culture is carried out such that cultureware housing cells in a liquid culture medium is rocked in the direction of a plane substantially vertical to the horizontal plane. The speed of rocking has no particular limitation and may be, for example, from 2 to 50 times (one back and forth movement is counted as one time) per minute or from 4 to 25 times per minute. The angle of rocking has no particular limitation and may be, for example, from 0.1 to 20 degrees or from 2 to 10 degrees. Setting the rocking culture condition to this range enables cell aggregates with an appropriate size to be produced.

Further, the culture may be mixed by movement in which the above rotary shaking and rocking are combined.

Culture using spinner flask-shaped cultureware in which mixing blades are placed may be carried out. During this culture, the liquid culture medium is mixed by the mixing blades. The speed of rotation and the volume of culture medium are not particularly limited. When commercially available spinner flask-shaped cultureware is used, the culture medium volume recommended by the manufacturer may be suitably used. The speed of rotation has no particular limitation and may be, for example, 10 rpm or more and 100 rpm or less.

The seeding density (i.e., the cell density at the start of suspension culture) of cells cultured in suspension in a liquid culture medium may be adjusted appropriately. The lower limit may be, for example, 0.01×10⁵ cells/ml, 0.1×10⁵ cells/ml, or 1×10⁵ cells/ml. The upper limit of the seeding density may be, for example, 20×10⁵ cells/ml or 10×10⁵ cells/ml. When the seeding density is within this range, cell aggregates with an appropriate size are likely to be formed.

The volume of culture medium during suspension culture may be appropriately adjusted depending on cultureware used. When a 12-well plate (with a bottom area per well of 3.5 cm² in a flat view) is used, for example, the volume may be 0.5 ml/well or more, 1.5 ml/well or more, or 1 ml/well. When a 6-well plate (with a bottom area per well of 9.6 cm² in a flat view) is used, for example, the volume may be 1.5 mL/well or more, 2 mL/well or more, or 3 mL/well. The volume may be 6.0 mL/well or less, 5 mL/well or less, or 4 mL/well or less. When a 125-mL Erlenmeyer flask (an Erlenmeyer flask with a volume of 125 mL) is used, for example, the volume may be 10 mL/flask or more, 15 mL/flask or more, 20 mL/flask or more, 25 mL/flask or more, 20 mL/flask or more, 25 mL/flask or more, or 30 mL/flask or more. The volume may be 50 mL/flask or less, 45 mL/flask or less, or 40 mL/flask or less. When a 500-mL Erlenmeyer flask (an Erlenmeyer flask with a volume of 500 mL) is used, for example, the volume may be 100 mL/flask or more, 105 mL/flask or more, 110 mL/flask or more, 115 mL/flask or more, or 120 mL/flask or more. The volume may be 150 mL/flask or less, 145 mL/flask or less, 140 mL/flask or less, 135 mL/flask or less, 130 mL/flask or less, or 125 mL/flask or less. When a 1000-mL Erlenmeyer flask (an Erlenmeyer flask with a volume of 1000 mL) is used, for example, the volume may be 250 mL/flask or more, 260 mL/flask or more, 270 mL/flask or more, 280 mL/flask or more, or 290 mL/flask or more. The volume may be 350 mL/flask or less, 340 mL/flask or less, 330 mL/flask or less, 320 mL/flask or less, or 310 mL/flask or less. When a 2000-mL Erlenmeyer flask (an Erlenmeyer flask with a volume of 2000 mL) is used, for example, the volume may be 500 mL/flask or more, 550 mL/flask or more, or 600 mL/flask or more. The volume may be 1000 mL/flask or less, 900 mL/flask or less, 800 mL/flask or less, or 700 mL/flask or less. When a 3000-mL Erlenmeyer flask (an Erlenmeyer flask with a volume of 3000 mL) is used, for example, the volume may be 1000 mL/flask or more, 1100 mL/flask or more, 1200 mL/flask or more, 1300 mL/flask or more, 1400 mL/flask or more, or 1500 mL/flask or more. The volume may be 2000 mL/flask or less, 1900 mL/flask or less, 1800 mL/flask or less, 1700 mL/flask or less, or 1600 mL/flask or less. When a 2-L culture bag (a disposable culture bag with a volume of 2 L) is used, for example, the volume may be 100 mL/bag or more, 200 mL/bag or more, 300 mL/bag or more, 400 mL/bag or more, 500 mL/bag or more, 600 mL/bag or more, 700 mL/bag or more, 800 mL/bag or more, 900 mL/bag or more, or 1000 mL/bag or more. The volume may be 2000 mL/bag or less, 1900 mL/bag or less, 1800 mL/bag or less, 1700 mL/bag or less, 1600 mL/bag or less, 1500 mL/bag or less, 1400 mL/bag or less, 1300 mL/bag or less, 1200 mL/bag or less, or 1100 mL/bag or less. When a 10-L culture bag (a disposable culture bag with a volume of 10 L) is used, for example, the volume may be 500 mL/bag or more, 1 L/bag or more, 2 L/bag or more, 3 L/bag or more, 4 L/bag or more, or 5 L/bag or more. The volume may be 10 L/bag or less, 9 L/bag or less, 8 L/bag or less, 7 L/bag or less, 6 L/bag or less. When a 20-L culture bag (a disposable culture bag with a volume of 20 L) is used, for example, the volume may be 1 L/bag or more, 2 L/bag or more, 3 L/bag or more, 4 L/bag or more, 5 L/bag or more, 6 L/bag or more, 7 L/bag or more, 8 L/bag or more, 9 L/bag or more, or 10 L/bag or more. The volume may be 20 L/bag or less, 19 L/bag or less, 18 L/bag or less, 17 L/bag or less, 16 L/bag or less, 15 L/bag or less, 14 L/bag or less, 13 L/bag or less, 12 L/bag or less, or 11 L/bag or less. When a 50-L culture bag (a disposable culture bag with a volume of 50 L) is used, for example, the volume may be 1 L/bag or more, 2 L/bag or more, 5 L/bag or more, 10 L/bag or more, 15 L/bag or more, 20 L/bag or more, or 25 L/bag or more. The volume may be 50 L/bag or less, 45 L/bag or less, 40 L/bag or less, 35 L/bag or less, or 30 L/bag or less. When the volume of culture medium is within these ranges, cell aggregates with an appropriate size are likely to be formed.

The volume of cultureware used has no particular limitation and may be suitably selected. The area of the bottom of a portion housing a liquid culture medium may be determined in a flat view. The lower limit of the bottom area of the cultureware used may be, for example, 0.32 cm², 0.65 cm², 0.65 cm², 1.9 cm², 3.0 cm², 3.5 cm², 9.0 cm², or 9.6 cm². The upper limit of the bottom area of the cultureware used may be, for example, 1000 cm², 500 cm², 300 cm², 150 cm², 75 cm², 55 cm², 25 cm², 21 cm², 9.6 cm², or 3.5 cm².

Conditions (e.g., the temperature, culture period, CO₂ level) of cell suspension culture in the presence of the above lipid have no particular limitation. The culture temperature may be 20° C. or higher, 35° C. or higher, 45° C. or lower, 40° C. or lower, or 37° C. The culture period may be 0.5 hour or longer, 12 hours or longer, 7 days or shorter, 72 hours or shorter, 48 hours or shorter, or 24 hours or shorter. The CO₂ level during the culture may be 4% or higher, 4.5% or higher, 10% or lower, 5.5% or lower, or 5%. The suspension culture may be split. When the culture conditions are within these ranges, cell aggregates with an appropriate size are likely to be formed.

Cells used in suspension culture may be pre-cultured and maintained in accordance with a common procedure. The maintenance culture may be adherent culture in which cells are cultured in contact with a culture substrate (e.g., a support) or may be suspension culture in which cells are cultured in suspension in a culture medium. Cells under the maintenance culture are detached from a culture substrate or are detached from one another by using the above-mentioned detachment agent. The resulting cells are sufficiently dispersed and are then cultured in suspension. In order to disperse the cells, they are made to pass through a strainer and can be dispersed as single cells.

Cell aggregates that have been formed by suspension culture in the presence of the above lipid may be further cultured. Examples of a method for further culturing cell aggregates include a method comprising suspending and culturing cell aggregates in a liquid culture medium free of the kinase inhibitor. As the liquid culture medium used for this additional culture, substantially the same liquid culture medium except that it needs to be free of kinase inhibitor may be used. The conditions used for this additional culture may be substantially the same conditions as the above. In this additional culture, a culture medium may be changed in an appropriate frequency. The frequency of the medium change may vary depending on a type of the cells. The frequency of medium change operation may be once or more per 5 days, once or more per 4 days, once or more per 3 days, once or more per 2 days, or once or more per day. This frequency of the medium change is suitable when cell aggregates of pluripotent stem cells prepared in one or more embodiments of the present invention are cultured. The medium change procedure has no particular limitation. A procedure may comprise: collecting all the volume of cell aggregate-containing culture medium into a centrifuge tube; subjecting the tube to centrifugation or allowing the tube to stand for 5 min; keeping precipitated cell aggregates and removing the rest supernatant and thereafter; adding a fresh liquid culture medium; gently dispersing the cell aggregates and thereafter; and returning the liquid culture medium containing the dispersed cell aggregates to cultureware (e.g., a plate), so that the cell aggregates can be cultured continuously. The culture period of the additional culture has no particular limitation and may be from 3 to 7 days.

The wording “step of adding the lysophospholipid to prepare the liquid culture medium” in one or more embodiments of the present invention refers to addition of the lysophospholipid to the liquid culture medium in the above-described concentration or ratio. The lysophospholipid may be added to the liquid culture medium and the isolated cells may then be mixed therewith. Alternatively, the isolated cells may be mixed with the liquid culture medium and the lysophospholipid may then be added. The lysophospholipid may be added to the liquid culture medium and the isolated cells may be then mixed therewith. When the lysophospholipid is added to the liquid culture medium, a stabilizer may be added. The stabilizer has no particular limitation as long as the substance can contribute to, for example, stabilization of the lysophospholipid in a liquid culture medium, maintenance of its activity, and prevention of adsorption on cultureware. A protein (e.g., albumin), emulsifier, surfactant, amphiphilic substance, or polysaccharide compound (e.g., heparin), for example, may be used. The “step of adding the lysophospholipid to prepare the liquid culture medium” may comprise a step of freezing the liquid culture medium containing the lysophospholipid (optionally containing the above-described stabilizer) and a step of thawing the liquid culture medium.

### EXAMPLES

One or more embodiments of the present invention is further described in detail by referring to the following Examples. However, they are just examples and do not restrict the present invention.

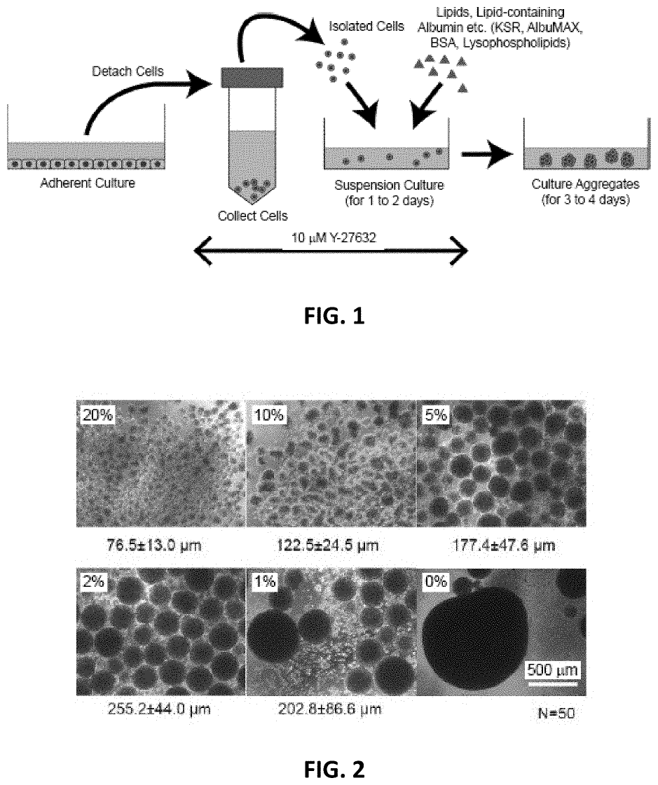

FIG. 1 schematically illustrates an outline of a protocol for producing cell aggregates as described in the following Examples. First, human iPS cells were subject to adherent culture and were collected as isolated cells. Next, the cells were cultured in suspension in a liquid culture medium. Then, the cells were cultured to grow aggregates. In the following description, the first day when the suspension culture started was designated as “Day 0”. The next day and later were each designated as “Day 1”, “Day 2”, “Day 3”, “Day 4”, or “Day 5”. The suspension culture was performed until Day 2 and the aggregates were grown for 3 days (i.e., from the start of the suspension culture to Day 5).

### Example 1: Maintenance Culture of Human iPS Cells

TkDN4-M cell line (Non-Patent Literature 1) was used as human iPS cells. The human iPS cells were seeded on cell culture dishes coated with Matrigel (Corning, Inc.) or Vitronectin (Life Technologies Japan Ltd.). A culture medium, which was mTeSR1 (STEMCELL Technologies, Inc.) or Essential 8™ (Life Technologies Japan Ltd.), was used for maintenance culture. As a cell detachment agent at the time of cell passage, TrypLE Select (Life Technologies Japan Ltd.) was used when the cells were cultured on Matrigel; and 0.02% EDTA (ethylene diamine tetraacetic acid) solution or Accutase (Life Technologies Japan Ltd.) was used when the cells were cultured on Vitronectin. In addition, when the cells were seeded, Y-27632 (Wako Pure Chemical Industries, Ltd.) at a concentration of 10 μM was added to a culture medium. The culture medium was changed every day. For experiments, human iPS cells (the number of passage was 50 or less) were used.

### Example 2: Formation of Aggregates of Human iPS Cells

Human iPS cells were treated with TrypLE Select or EDTA solution for 3 to 5 min and were detached. After dispersed as single cells, the cells were made to pass through a cell strainer with a pore size of 40 μm (Becton, Dickinson and Company). The resulting cells were suspended in Essential 8™ medium containing a final concentration of 10 μM Y-27632 (Wako Pure Chemical Industries, Ltd.). A portion thereof was then stained with trypan blue and the number of cells was counted. Suspensions containing 2×10⁵ cells per ml were prepared and different concentrations of KnockOut™ Serum Replacement (KSR; Life Technologies Japan Ltd.) were then added thereto. Each cell suspension was plated on a low-attachment 12-well plate (Nunc, Inc.) at a ratio of 1 ml/well. The cell-seeded plate was rotated along the horizontal plane on a rotary shaker (OPTIMA, Inc.) at a speed of 90 rpm in a circle with a shaking width (diameter) of 25 mm, so that the cells were subjected to rotary shaking culture. In this way, the cells were cultured in suspension under conditions at 5% CO₂ and 37° C. until Day 2. At Day 2 after the start of culture, micrographs were obtained.

Subsequently, the culture medium was replaced by KSR (lipid)- and Y-27632-free Essential 8™ medium. The cells were then subjected to rotary shaking culture under the same conditions for 3 days (from the start of the suspension culture to Day 5). The culture medium was changed every day during that period. A medium change procedure involves: collecting all the volume of the culture medium containing cell aggregates; and letting them stand for about 5 min to precipitate the cell aggregates. Then, the supernatant was removed; a fresh medium was added; the cell aggregates were gently resuspended; and the cells were reseeded on a low-adherence 12-well plate.

Micrographs of cell aggregates were taken at Day 5 after the start of culture (Day 3 of the aggregation culture), and the size of each cell aggregate was analyzed by image-analyzing software (e.g., image J) to determine the diameter of each cell aggregate. After culturing, the aggregates were suspended and treated in TrypLE select (Life Technologies Japan Ltd.) for 10 min under conditions at 5% CO₂ and 37° C., and pipetted to disperse the aggregates into single cells. Then, the resulting cells were stained with trypan blue to count the number of cells.

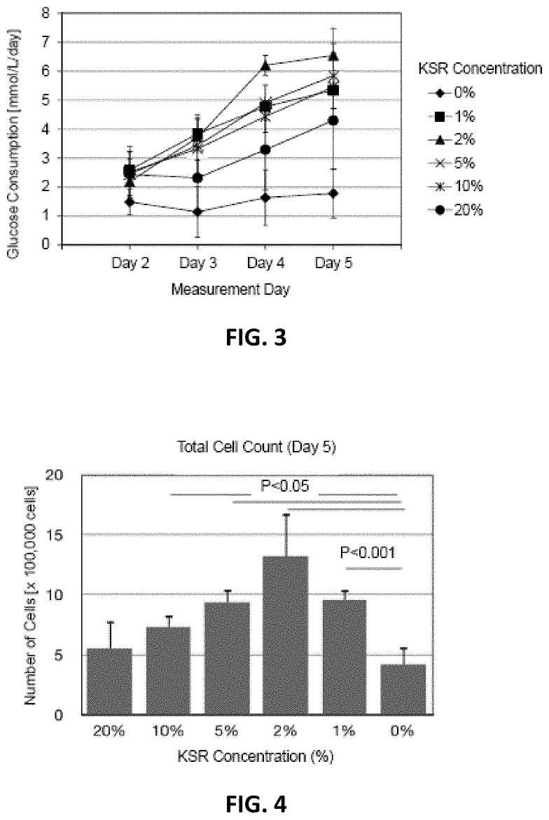

The above microscopic observation results demonstrated that under KSR-free conditions, large aggregates were formed, but when KSR was added, a large number of aggregates with a diameter of from 70 to 260 μm were formed (FIG. 2). As the concentration of KSR added increased, the size of each aggregate became smaller. This revealed that some component included in KSR exerted an effect of inhibiting cell aggregation. In addition, with respect to the aggregates formed, the glucose consumption and the number of cells (at Day 5) at each KSR concentration were investigated. The results (FIGS. 3 and 4; n=4 in both figures) demonstrated remarkable proliferation of cells when the concentration of KSR was from 1 to 10% (v/v) (when the content of lipid was from 0.00325 to 0.0325 mg/ml). This result demonstrated that efficient cell culture was made available when cells were cultured in such a condition that cell aggregates with a diameter of from 100 to 260 μm were formed. By contrast, excessive inhibition of cell aggregation clearly inhibited the proliferation of cells.

### Example 3: KSR Contained Component Having Effect of Inhibiting Aggregation

As components constituting KSR, components shown in FIG. 5 have been reported (Non-Patent Literature 2).

It was examined which protein-based component (i.e., AlbuMAX™, BSA, insulin, transferrin), which might seem to exert the effect of inhibiting aggregation, among the above components, exerted an effect of inhibiting the aggregation of human iPS cells when added.

Specifically, the following procedure was conducted: the same protocol as in Example 2 was repeated except that one of the following factors instead of KSR in Example 2 was each added, including various concentrations of lipid-rich albumin AlbuMAX™ II (Life Technologies Japan Ltd.), typical bovine serum albumin (BSA; Sigma-Aldrich Co. LLC.), insulin (Sigma-Aldrich Co. LLC.), and transferrin (Sigma-Aldrich Co. LLC.). That is, human iPS cells were subjected to rotary shaking culture (the cells were cultured in suspension in the presence of each factor for 2 days, followed by culture medium change and subsequent suspension culture for 3 days). Micrographs were obtained at Day 2 after the start of culture under conditions in which AlbuMAX™ II or BSA was added. Micrographs were obtained at Day 1 after the start of culture under conditions in which insulin or transferrin was added. The concentration of each factor added in Example 3, % (w/v), was represented in weight (g) per 100 ml of a liquid culture medium.

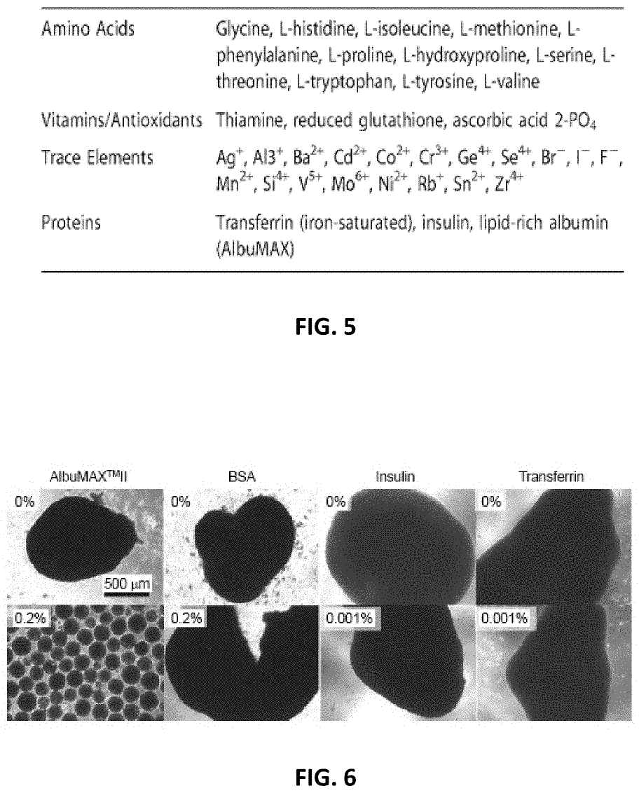

The results demonstrated that lipid-free insulin and transferrin caused large aggregates to be formed; and that an addition of 0.2% (w/v) AlbuMAX™ II (with a lipid content of 0.013 mg/ml) caused more marked inhibition of aggregation than an addition of 0.2% (w/v) BSA. This revealed that the lipids contained in AlbuMAX™ II, namely the lipids that can bind to albumin, inhibited the aggregation (FIG. 6).

### Example 4: Undifferentiated State of Human iPS Cells Forming Aggregates

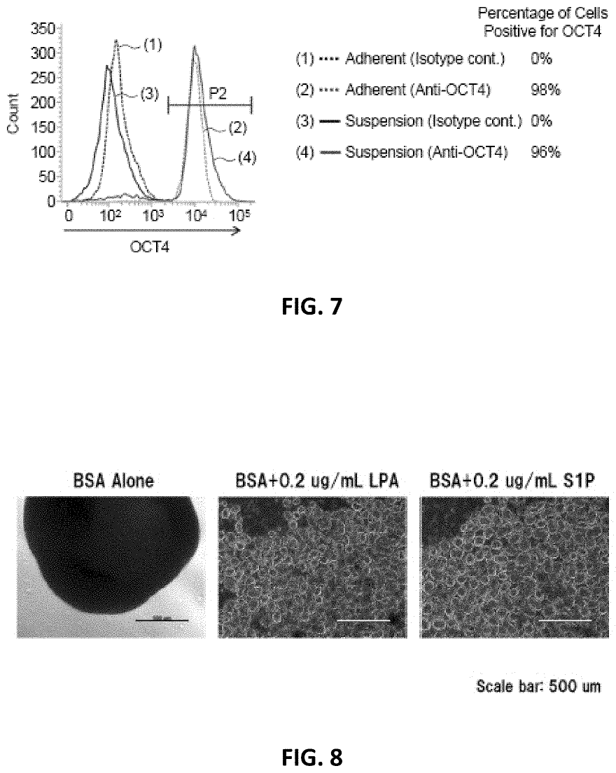

In Example 3, human iPS cells were subjected to culture conditions containing 0.2% (w/v) AlbuMAX™ II to form aggregates. These human iPS cell-derived aggregates were dispersed using Accutase (Life Technologies Japan Ltd.) and were washed with PBS (phosphate buffered saline). Next, the resulting cells were fixed with 4% PFA (paraformaldehyde) at room temperature for 20 min, then washed 3 times with PBS, and permeabilized with cold methanol at −20° C. overnight. After washed 3 times with PBS, the cells were blocked with 3% FBS (fetal calf serum)/PBS and stained using a fluorescently labeled anti-OCT4 antibody (Cat. No. 653703, Biolegend, Inc.) at 4° C. for 1 h. After washed once with 3% FBS (fetal calf serum)/PBS, the cells were made to pass through a cell strainer. The resulting cells were analyzed on FACSVerse (Becton, Dickinson and Company). The results demonstrated that 96% or more of human iPS cells that formed aggregates as well as human iPS cells during conventional adherent culture expressed OCT4, which is an undifferentiation marker; and that the human iPS cells forming aggregates remained undifferentiated (FIG. 7).

### Example 5: Effect of Phospholipid on Inhibiting Cell Aggregation

How a phospholipid affected the formation of cell aggregates was analyzed.

### (Protocol)

Human iPS cells that had been cultured using the protocol of Example 1 were treated with TrypLE Select, EDTA solution, or Accutase for 3 to 5 min and were detached. After dispersed as single cells, the cells were made to pass through a cell strainer with a pore size of 40 μm (Becton, Dickinson and Company) to monodisperse as single cells. The resulting cells were suspended in Essential 8™ medium containing a final concentration of 10 μM Y-27632 (Wako Pure Chemical Industries, Ltd.). A portion thereof was then stained with trypan blue and the number of cells was counted. Suspensions containing 2×10⁵ cells per ml were prepared. To each cell suspension were added lipid-free bovine serum albumin (BSA-ff; CultureSure albumin, Wako Pure Chemical Industries, Ltd.) at a final concentration of 5 mg/mL and Y-27632 (Wako Pure Chemical Industries, Ltd.) at a final concentration of 10 μM. In addition, the following lipids were each added to the cell suspension and the cells were subjected to rotary shaking culture for 1 day under the same conditions as in Example 2 to culture human iPS cells in suspension. After one day of the suspension culture, the cells were observed under a microscope.

The lipids and additives used in this study are as follows:
LPA (Lysophosphatidic acid) (1-O-9Z-octadecenoyl-sn-glyceryl-3-phosphoric acid, a sodium salt, Cat. No. 62215, Cayman, Inc.; with an oleyl group at sn-1 position).

S1P (Sphingosine-1-phosphoric acid) (2S-amino-1-(dihydrogen phosphate)-4E-octadecene-1,3R-diol, Cat. No. 62570, Cayman, Inc.)

LPA at 0.2 μg/mL or S1P at 0.2 μg/mL was added to the human iPS cell suspension.

A control test was conducted using the cell suspension prepared under the same conditions as above except that the above lipids were not added.

### (Results)

FIG. 8 shows micrographs after the above suspension culture. The observation results demonstrated that in the control test, large aggregates (with a diameter of 1 mm or larger) were formed; and when the cell suspension contained 0.2 μg/mL of LPA (lysophosphatidic acid) or S1P (sphingosine-1-phosphoric acid), a large number of spherical cell aggregates with a substantially uniform size of about 100 μm were formed.

### Example 6: Concentration of LPA or S1P Added and Size of Aggregates

In Example 5, LPA and S1P were found to exert an ability to inhibit cell aggregation. Here, cell suspensions containing different concentrations of LPA or S1P added were used to perform suspension culture.

### (LPA Concentration)

Human iPS cell suspensions were prepared using substantially the same protocol as in Example 5. LPA was added as a sodium salt at a concentration of 0.00128 μg/mL, 0.0064 μg/mL, 0.032 μg/mL, 0.16 μg/mL, 0.8 μg/mL, 4 μg/mL, 20 μg/mL, or 100 μg/mL. To each suspension were added BSA-ff at a final concentration of 5 mg/mL and Y-27632 (Wako Pure Chemical Industries, Ltd.) at a final concentration of 10 μM. The cells were cultured in suspension for 1 day under the same conditions as in Example 5 and then observed under a microscope.

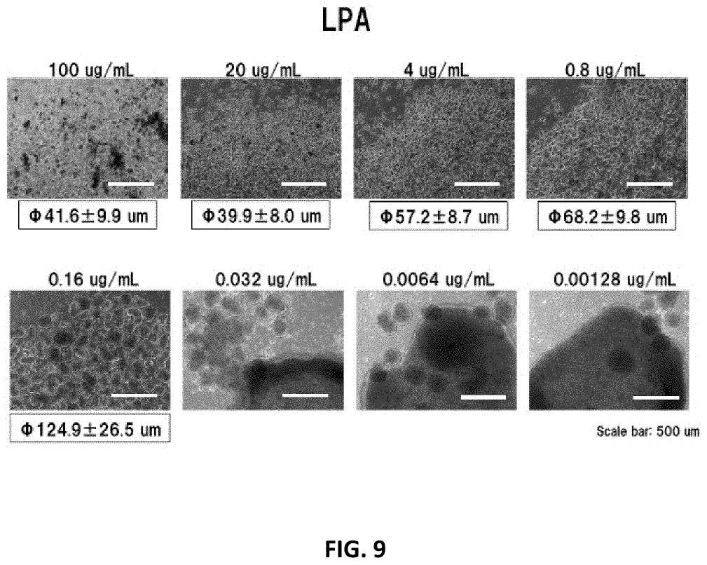

FIG. 9 shows the observation results. The size of each cell aggregate changed depending on the concentration of LPA. When the concentration of LPA was from 0.16 to 100 μg/mL, cell aggregates with a substantially uniform size were formed. When the concentration of LPA is 0.032 μg/mL or less, large cell aggregates with a diameter of 1 mm or larger were formed.

Inspected were 30 cell aggregates on an observation image obtained when the concentration of LPA was from 0.16 to 100 μg/mL. While being compared using a micrograph scale, the width (referred to as “φ”) of the widest portion of each cell aggregate was measured to calculate an average±standard deviation.

When the concentration of LPA was 0.16 μg/mL, φ=124.9±26.5 μm; at 0.8 μg/mL, φ=68.2±9.8 μm; at 4 μg/mL, φ=57.2±8.7 μm; at 20 μg/mL, φ=39.9±8.0 μm; and at 100 μg/mL, φ=41.6±9.9 μm.

### (S1P Concentration)

Human iPS cell suspensions were prepared using substantially the same protocol as in Example 5. S1P was added as a free form at a concentration of 0.00128 μg/mL, 0.0064 μg/mL, 0.032 μg/mL, 0.16 μg/mL, 0.8 μg/mL, 4 μg/mL, 20 μg/mL, or 100 μg/mL. To each suspension were added BSA-ff at a final concentration of 5 mg/mL and Y-27632 (Wako Pure Chemical Industries, Ltd.) at a final concentration of 10 μM. The cells were cultured in suspension for 1 day under the same conditions as in Example 5 and then observed under a microscope.

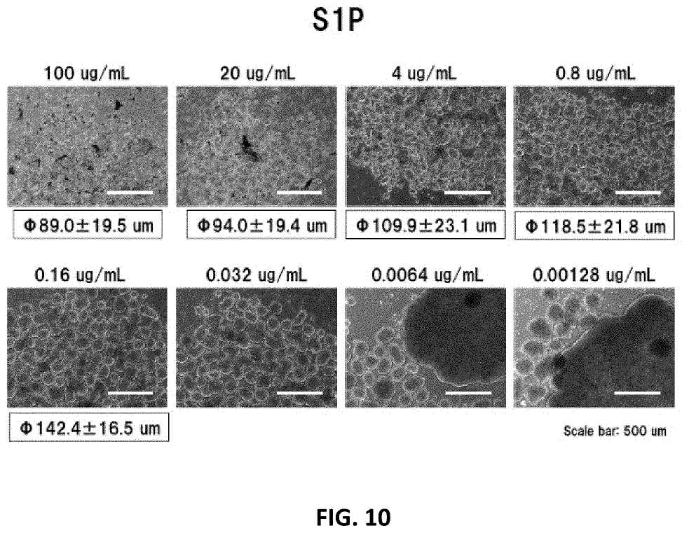

FIG. 10 shows the observation results. The size of each cell aggregate changed depending on the concentration of S1P. When the concentration of S1P was from 0.032 to 100 μg/mL, cell aggregates with a substantially uniform size were formed. When the concentration of S1P is 0.0064 μg/mL or less, large cell aggregates with a diameter of 1 mm or larger were formed.

Inspected were 30 cell aggregates on an observation image obtained when the concentration of S1P was from 0.032 to 100 μg/mL. While being compared using a micrograph scale, the width (referred to as “φ”) of the widest portion of each cell aggregate was measured to calculate an average±standard deviation.

When the concentration of S1P was 0.16 μg/mL, φ=142.4±16.4 μm; at 0.8 μg/mL, φ=118.5±21.8 μm; at 4 μg/mL, φ=109.9±23.1 μm; at 20 μg/mL, φ=94.0±19.4 μm; and at 100 μg/mL, φ=89.0±19.5 μm.

### Example 7: Effect of the Presence of LPA or S1P on Cell Proliferation Potential and Undifferentiated State

Human iPS cells were cultured in suspension under culture conditions in the presence of LPA or S1P at different concentrations. Next, the glucose consumption, the total cell count, and the percentage of cells positive for undifferentiation markers were determined. How these additives affected the cells was analyzed.

### (Protocol)

Human iPS cell suspensions were prepared using substantially the same protocol as in Example 5. To each cell suspension were added LPA or S1P at a final concentration of 0.2 μg/mL or 1 μg/mL, and BSA-ff at a final concentration of 5 mg/mL and Y-27632 (Wako Pure Chemical Industries, Ltd.) at a final concentration of 10 μM. As a control, cell suspensions were prepared using the same protocol as in Example 5. That is, prepared were the cell suspensions solely containing BSA-ff at a final concentration of 5 mg/mL and Y-27632 at a final concentration of 10 μM.

The above cell suspensions were subjected to suspension culture for 2 days under the same conditions as in Example 5. At Day 2 of culture, the culture medium was changed every day with Essential 8™ culture medium supplemented with 5 mg/mL BSA-ff. At Days 2, 3, 4, and 5, the concentration of glucose in the culture supernatant was measured to calculate glucose consumption. In addition, at Day 5 of culture, cell aggregates were collected, dispersed using Accutase, and suspended in Essential 8™ culture medium supplemented with 5 mg/mL BSA-ff. A portion of each cell suspension was stained with trypan blue and the number of cells was counted. The above cell suspensions were centrifuged at 300 g for 5 min, the supernatant was then removed, and the cells were washed with PBS (phosphate buffered saline). Next, the cells were fixed with 4% PFA (paraformaldehyde) at room temperature for 20 min, then washed 3 times with PBS, and permeabilized with cold methanol at −20° C. overnight. After washed 3 times with PBS, the cells were blocked with 3% FBS (fetal calf serum)/PBS and stained using a fluorescently labeled anti-SOX2 antibody (Cat. No. 656110, Biolegend, Inc.) and a fluorescently labeled anti-OCT4 antibody (Cat. No. 653703, Biolegend, Inc.) at 4° C. for 1 h. After washed once with 3% FBS (fetal calf serum)/PBS, the cells were made to pass through a cell strainer. The resulting cells were analyzed on FACSVerse.

The following procedure was used to measure glucose consumption. Specifically, the culture supernatant was recovered at medium change and a bioanalyzer (Y512950), manufactured by YSI, Inc., was used to measure the remaining glucose amount. In this way, the glucose consumption was calculated.

A total cell count was measured at Day 5 of culture. The following procedure was used to measure the total cell count. Specifically, the cell aggregates that had been formed were treated with TrypLE Select for 5 to 10 min, pipetted using a blue tip to monodisperse as cells, and stained with trypan blue. After that, the number of cells was counted using a hemocytometer to determine the total cell count.

### (Results)

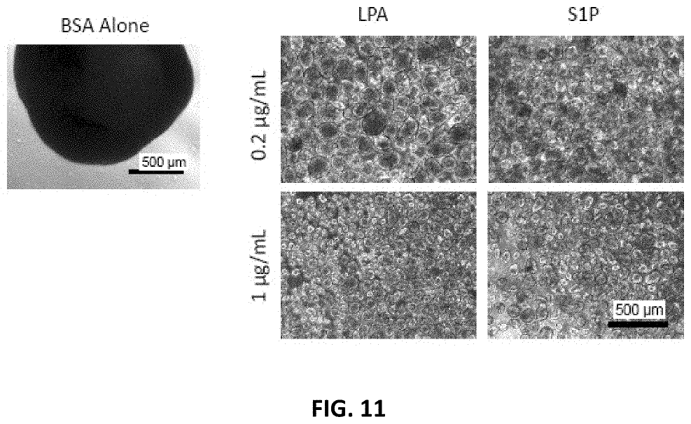

The pictures of FIG. 11 are micrographs obtained at Day 2 after the start of culture. When LPA and S1P were each added and tested, cell aggregates with an appropriate size (with a diameter of 500 μm or less) were formed. By contrast, when BSA-ff alone was added and tested, large cell aggregates (with a diameter of 1 mm or more) were formed.

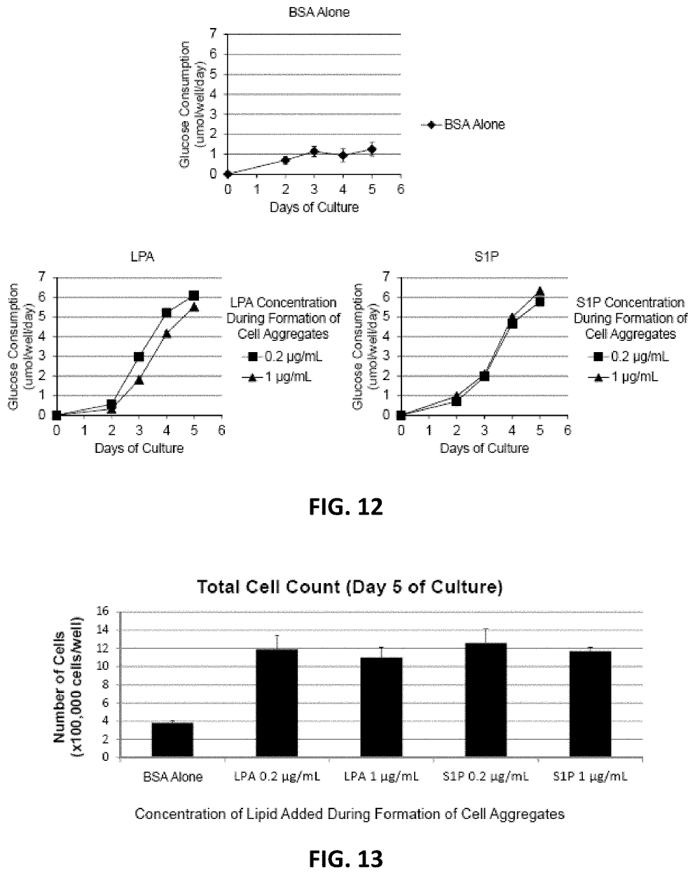

FIG. 12 shows the results of measuring glucose consumption. FIG. 13 shows the total cell count at Day 5 of culture. The glucose consumption and the number of cells were larger in the case of the addition of LPA or S1P than the case of the addition of BSA-ff alone. This revealed that the cells proliferated remarkably when LPA or S1P was added.

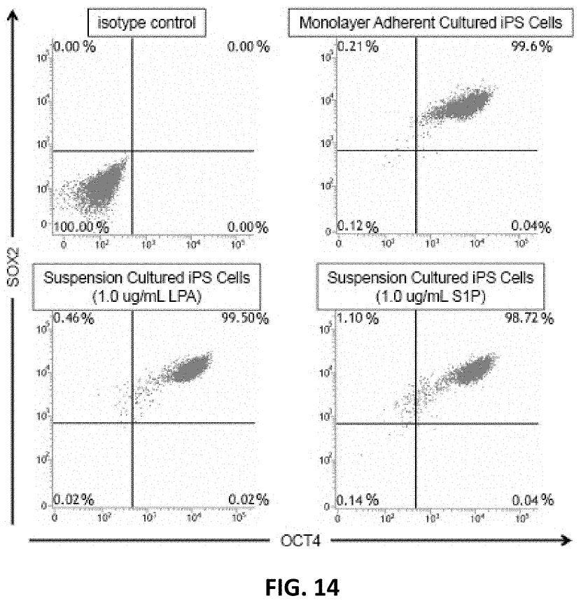

FIG. 14 shows the results of measuring the percentage of cells positive for undifferentiation markers. When the cells were cultured as a suspension containing 1 μg/mL of LPA or S1P, 95% or more of the cells expressed undifferentiation markers OCT4 and SOX2, which is similar to cells in monolayer adherent culture. This verified that the human iPS cell aggregates that had been formed by the addition of the lipid remained undifferentiated.

### Example 8: Examination of how Culture can be Split and Scaled Up

Human iPS cell suspensions were prepared using substantially the same protocol as in Example 5. AlbuMAX™ II was added at a final concentration of 5 mg/mL, BSA-ff was added at a final concentration of 5 mg/mL, and Y-27632 was added at a final concentration of 10 μM. The volume of culture medium was set to 4 mL per well, and the cells were seeded on a 6-well plate (Sumitomo Bakelite Co., Ltd.) at a cell density of 2×10⁵ cells per ml. The plate was rotated on a rotary shaker (OPTIMA, Inc.) at a speed of 90 rpm and cells were cultured under conditions at 5% CO₂ and 37° C. for 2 days to form aggregates. Then, the culture medium was replaced every day for 4 days by Essential 8™ culture medium containing a final concentration of 0.5 mg/mL AlbuMAX™ II and a final concentration of 5 mg/mL BSA-ff. The following procedure was used to split cells at 6 days after the cell seeding. Cell aggregates were collected and washed once with PBS. Next, the cell aggregates were subjected to Accutase treatment at 37° C. for 10 min to disperse the cells. Then, Essential 8™ culture medium containing a final concentration of 5 mg/mL BSA-ff was added. After the mixture was centrifuged at 300 g for 3 min and the supernatant was removed, human iPS cell suspensions (supplemented with a final concentration of 5 mg/mL AlbuMAX™ II, a final concentration of 5 mg/mL BSA-ff, and a final concentration of 10 μM Y-27632) were likewise prepared. The cells were seeded in a 1-L Erlenmeyer flask (Corning, Inc., product No. 431147) such that the volume of culture medium was 300 mL per flask and the cell density was 2×10⁵ cells per ml. The cells were cultured like the case of the above 6-well plate. At 5 days after the seeding, human iPS cell suspensions (supplemented with a final concentration of 5 mg/mL AlbuMAX™ II, a final concentration of 5 mg/mL BSA-ff, and a final concentration of 10 μM Y-27632) were likewise prepared. The cells were seeded and split in a 3-L Erlenmeyer flask (Corning, Inc., product No. 431252) such that the volume of culture medium was 1.6 L per flask and the cell density was 2×10⁵ cells per ml. The speed of rotation was set to 70 rpm and substantially the same procedure as above was used to culture the cells. For each culture volume (at 4-mL scale, 300-mL scale, and 1.6-L scale), micrographs of cell aggregates at 1, 5, or 6 days after the seeding were taken. In addition, the same procedure as in Example 2 was used to count the number of cells at the final day of culture. Also, the same procedure as in Example 7 was used to analyze the percentage of cells positive for the undifferentiation markers. As a control, adherent cultured cells as obtained using the procedure of Example 1 were used.

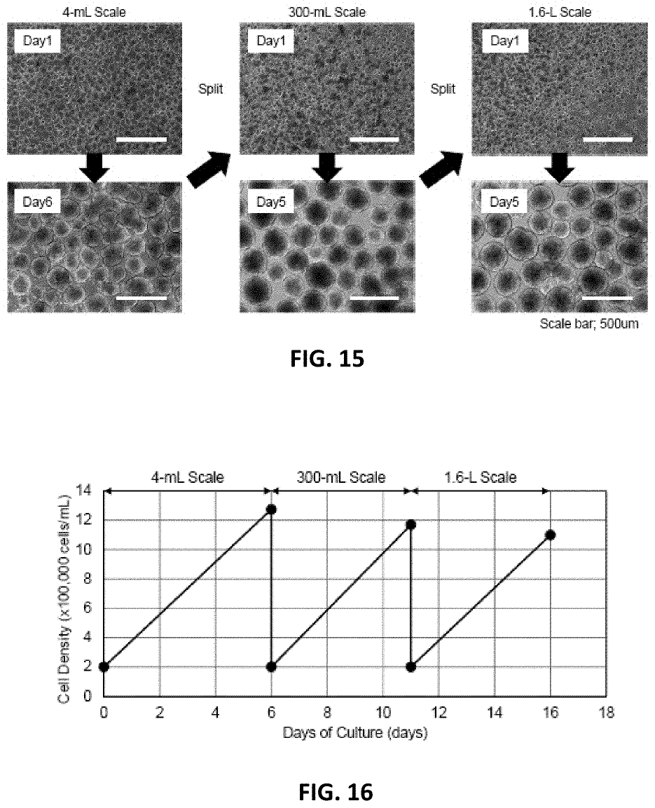

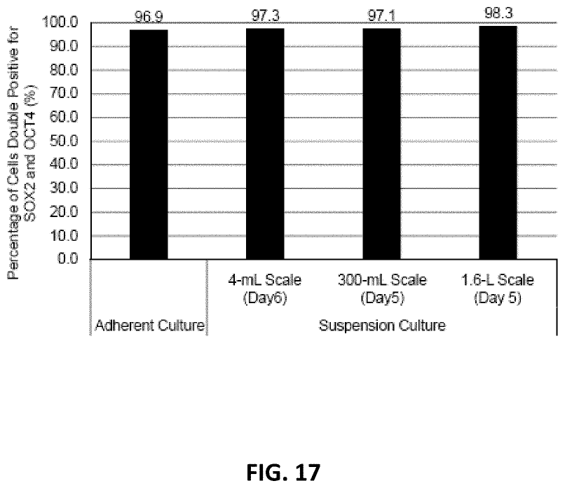

The above microscopic observation results have demonstrated that regardless of the culture scale, cell aggregates with a substantially uniform size can be likewise formed and that the cells can proliferate while remaining undifferentiated (FIGS. 15, 16, and 17).

All the publications, patents, and patent applications cited herein are incorporated herein by reference in its entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method for producing cell aggregates, comprising culturing cells while suspending the cells in a liquid culture medium comprising a lysophospholipid.

2. The method according to claim 1, wherein the liquid culture medium comprises the lysophospholipid in an amount greater than 0.0064 μg/mL to 100 μg/mL.

3. The method according to claim 1, further comprising preparing the liquid culture medium by at least one of adding the lysophospholipid to the liquid culture medium and generating the lysophospholipid through an enzymatic reaction in the liquid culture medium.

4. The method according to claim 1, wherein the cells are cells isolated from an adherent culture or a suspension culture.

5. The method according to claim 1, wherein the lysophospholipid is at least one of lysophosphatidic acid and sphingosine-1-phosphoric acid.

6. The method according to claim 1, wherein the liquid culture medium comprises at least one selected from the group consisting of L-ascorbic acid, insulin, transferrin, selenium, and sodium bicarbonate.

7. The method according to claim 1, wherein the liquid culture medium comprises a growth factor.

8. The method according to claim 7, wherein the growth factor is at least one of FGF2 and TGF-β1.

9. The method according to claim 1, wherein the liquid culture medium comprises a ROCK inhibitor.

10. The method according to claim 9, wherein the ROCK inhibitor is Y-27632.

11. The method according to claim 1, wherein the cells are pluripotent stem cells.

12. A composition comprising a lysophospholipid, wherein the composition is a liquid culture medium or a composition added to a liquid culture medium.

13. The composition according to claim 12, comprising the lysophospholipid in an amount greater than 0.0064 μg/mL to 100 μg/mL.

14. The composition according to claim 12, further comprising a growth factor.

15. The composition according to claim 14, wherein the growth factor is at least one of FGF2 and TGF-β1.

16. The composition according to claim 12, further comprising a ROCK inhibitor.

17. The composition according to claim 16, wherein the ROCK inhibitor is Y-27632.

18. The composition according to claim 12, wherein the lysophospholipid is at least one of lysophosphatidic acid and sphingosine-1-phosphoric acid.
